SEQUENCE LISTING (25 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 8 aa, synthetic construct
  VARIANT 1: note = L, A, V, or G
  VARIANT 4: note = F, A, V, or G
  VARIANT 8: note = L, A, V, or G
XDSXKEEX

SEQ ID NO: 3 - protein, 8 aa, synthetic construct
  VARIANT 1: note = A, V, G, or any other natural or synthetic or unnatural or modified amino acid, but not L
XDSFKEEL

SEQ ID NO: 4 - protein, 8 aa, synthetic construct
  VARIANT 4: note = A, V, G, or any other natural or synthetic or unnatural or modified amino acid, but not F
LDSXKEEL

SEQ ID NO: 5 - protein, 8 aa, synthetic construct
  VARIANT 8: note = A, V, G, or any other natural or synthetic or unnatural or modified amino acid, but not L
LDSFKEEX

SEQ ID NO: 6 - protein, 8 aa, synthetic construct
  VARIANT 1: note = L, A, V, or G, or any other natural or synthetic or unnatural or modified amino acid
  VARIANT 4: note = F, A, V, or G, or any other natural or synthetic or unnatural or modified amino acid
XDSXKEEL

SEQ ID NO: 7 - protein, 8 aa, synthetic construct
  VARIANT 1: note = L, A, V, or G, or any other natural or synthetic or unnatural or modified amino acid
  VARIANT 8: note = L, A, V, or G, or any other natural or synthetic or unnatural or modified amino acid
XDSFKEEX

SEQ ID NO: 8 - protein, 8 aa, synthetic construct
  VARIANT 4: note = F, A, V, or G, or any other natural or synthetic or unnatural or modified amino acid
  VARIANT 8: note = L, A, V, or G, or any other natural or synthetic or unnatural or modified amino acid
LDSXKEEX

SEQ ID NO: 9 - protein, 8 aa, synthetic construct
ADSAKEEA

SEQ ID NO: 10 - protein, 8 aa, unidentified
  source 1..8: note = alpha-7 nicotinic acetylcholine receptor sequence
LAKILEEV

SEQ ID NO: 11 - DNA, 38 bp, synthetic construct
  source 1..38: mol_type = other DNA
tctagaggatcgtcgccaccatgcgctgctcgccggga

SEQ ID NO: 12 - DNA, 45 bp, synthetic construct
  source 1..45: mol_type = other DNA
ctagactcgatgatcagttactacgcaaagtctttggacacggcc

SEQ ID NO: 13 - DNA, 24 bp, synthetic construct
  source 1..24: mol_type = other DNA
taactgatcatcgagtctagaggg

SEQ ID NO: 14 - DNA, 20 bp, synthetic construct
  source 1..20: mol_type = other DNA
ggtggcgacgatcctctaga

SEQ ID NO: 15 - DNA, 38 bp, synthetic construct
  source 1..38: mol_type = other DNA
tgtgctgaacgatatcctgtctagactggacaaggtgg

SEQ ID NO: 16 - DNA, 42 bp, synthetic construct
  source 1..42: mol_type = other DNA
ggcctcttccttggcagagtcggcctcaggttgcagagggtc

SEQ ID NO: 17 - DNA, 52 bp, synthetic construct
  source 1..52: mol_type = other DNA
gccgactctgccaaggaagaggccgacaagtactttaaaaaccacaccagcc

SEQ ID NO: 18 - DNA, 38 bp, synthetic construct
  source 1..38: mol_type = other DNA
ctatgaccatgattacgccaagcttgggctgcaggtcg

SEQ ID NO: 19 - skipped

SEQ ID NO: 20 - protein, 8 aa, synthetic construct
  VARIANT 2: note = any natural or synthetic or unnatural or modified amino acid, but not D
LXSFKEEL

SEQ ID NO: 21 - protein, 8 aa, synthetic construct
  VARIANT 3: note = any natural or synthetic or unnatural or modified amino acid, but not S
LDXFKEEL

SEQ ID NO: 22 - protein, 8 aa, synthetic construct
  VARIANT 6: note = any natural or synthetic or unnatural or modified amino acid, but not E
LDSFKXEL

SEQ ID NO: 23 - protein, 8 aa, synthetic construct
  VARIANT 7: note = any natural or synthetic or unnatural or modified amino acid, but not E
LDSFKEXL

SEQ ID NO: 24 - protein, 4 aa, synthetic construct
RRAR

SEQ ID NO: 25 - protein, 8 aa, Severe acute respiratory syndrome coronavirus 2
LDSFKEEL

(19) United States
(12) Patent Application Publication
(10) Pub. No.: US 2024/0100152 A1
(43) Pub. Date: Mar. 28, 2024

(54) MODIFIED POLYNUCLEOTIDES ENCODING THE SARS-COV-2 SPIKE PROTEIN FOR SAFER DESIGNS OF CORONAVIRUS VACCINES

(71) Applicant: University of Pittsburgh - Of the Commonwealth System of Higher Education, Pittsburgh, PA (US)

(72) Inventors: Pei Tang, Pittsburgh, PA (US); Tommy Stevens Tillman, Pittsburgh, PA (US); Yan Xu, Pittsburgh, PA (US)

(21) Appl. No.: 18/461,955

(22) Filed: Sep. 6, 2023

Related U.S. Application Data
(60) Provisional application No. 63/403,935, filed on Sep. 6, 2022.

(51) Int. Cl. A61K 39/215; A61P 37/04

(52) U.S. Cl. CPC A61K 39/215; A61P 37/04; A61K 2039/55555

(57) ABSTRACT

Recombinant polynucleotides including a nucleic acid sequence encoding an engineered SARS-CoV-2 spike protein (S12) or an immunogenic fragment thereof, in which the engineered spike protein or immunogenic fragment includes one or more mutations in the S2 segment of the S12 ectodomain. Vaccines, pharmaceutical compositions, and methods of use of the polynucleotides, vaccines, and pharmaceutical compositions in treating, alleviating, or managing SARS-CoV-2 infection and/or one or more symptoms thereof.

### CROSS-REFERENCE TO RELATED APPLICATIONS

This application claims the benefit of and priority to U.S. Provisional Application No. 63/403,935, filed on Sep. 6, 2022, the entire disclosure of which is hereby incorporated by reference in its entirety for all purposes.

### STATEMENT REGARDING FEDERALLY SPONSORED RESEARCH OR DEVELOPMENT

This invention was made with government support under grant number DA046939 awarded by the National Institutes of Health (NIH). The Government has certain rights in the invention.

### SEQUENCE LISTING

This application contains a Sequence Listing which has been submitted electronically and is hereby incorporated by reference in its entirety. Said XML copy, created on Aug. 31, 2023, is named UPB-026_SL.xml, and is 30,613 bytes in size.

### FIELD OF THE DISCLOSURE

The invention relates to variants of SARS-CoV-2 spike protein (S12), vaccines, pharmaceutical compositions, and therapeutic methods useful for the prevention and/or treatment of viral infections such as COVID-19.

### BACKGROUND

SARS-CoV-2 infects human cells through its spike protein that comprises two major domains, S1 and S2. S1 is responsible for recognition and binding to the angiotensin-converting enzyme 2 (ACE2) receptor on the surface of target cells, while S2 mediates fusion of viral and host cell membranes. The spike protein facilitates virus cell entry that leads to an acute infection known as COVID-19 (Brodin, 2021; Helms et al., 2020; Moore and June, 2020; Taquet et al., 2022). A subpopulation of patients experiences long-term sequelae from the infection, known as long COVID, which is characterized by a wide range of health issues (Huang et al., 2021; Nalbandian et al., 2021; Phillips and Williams, 2021; Taquet et al., 2022), including brain fog and other neurological and psychiatric problems (Graham et al., 2021; Huang et al., 2021; Nalbandian et al., 2021; Taquet et al., 2021; Taquet et al., 2022). The precise cause of long COVID is yet to be determined, but it is unlikely through a single mechanism. Direct action of the spike protein, S12, has been considered as a potential cause for some of the detrimental effects of SARS-CoV-2 (Theoharides, 2022). Immunoreactive S12, suspected to contribute to cardiovascular disease independent of viral infection, was found circulating in the blood of COVID-19 patients (Avolio et al., 2021). S12 can act alone or in conjunction with other mediators on target cells, stimulate different cell types, damage the integrity of the blood-brain barrier, and contribute to the pathogenesis of long COVID (Kumar et al., 2021; Perico et al., 2022; Robles et al., 2022; Singh et al., 2022; Theoharides, 2022). These findings undoubtedly implicate S12 as an inducer of cellular dysfunction.

The α7 nicotinic acetylcholine receptor (α7nAChR) was linked to COVID-19 pathophysiology at the early stage of the pandemic (Changeux et al., 2020). α7nAChR forms homo-pentameric ligand gated ion channels that mediate synaptic transmission in the central and peripheral nervous systems (Albuquerque et al., 2009; Couturier et al., 1990). It is involved in cognitive function, mental health, and neurodegenerative diseases (Letsinger et al., 2022). α7nAChR is also a major player in the cholinergic anti-inflammatory pathway (Pavlov and Tracey, 2005; Pavlov et al., 2003), which attenuates proinflammatory cytokine production and minimizes tissue and organ injury during inflammation. The α7nAChR agonists, such as nicotine, are essential for initiating the cholinergic anti-inflammatory pathway and effective in reducing macrophage cytokine production and inflammation (Song et al., 2018; Ulloa, 2005). Therapeutic implications of cholinergic signaling in acute and chronic pathology, including a therapeutic avenue for treating COVID-19, have been supported by human data and animal studies (Fudim et al., 2020; Schloss et al., 2022; Yang et al., 2022). α7nAChR is widely expressed across the human body in both neuronal and non-neuronal cells (Bencherif et al., 2011; Corradi and Bouzat, 2016; Letsinger et al., 2022; Pavlov and Tracey, 2017; Schloss et al., 2022; Wang et al., 2003). In the brain, it is expressed on both pre-and postsynaptic membranes, and particularly in regions implicated in cognitive function, such as the hippocampus and cortex (Gotti et al., 2006b; Lendvai et al., 2013). α7nAChR is also expressed in immune cells, such as macrophages, that form the basis for some of the known α7nAChR-mediated anti-inflammatory effects (Wang et al., 2003). A deficiency of functional α7nAChR is implicated in neuropsychic diseases and disrupts the cholinergic anti-inflammatory pathway (Cheng and Yakel, 2015; Corradi and Bouzat, 2016; Freedman et al., 1995; Gotti et al., 2006a; Koukouli and Maskos, 2015; Lange et al., 1993; Mizrachi et al., 2021). The current disclosure provides variants of the SARS CoV-2 spike protein ectodomain (S12) in which S2 segment when mutated abolishes the effects of the wild type S12 to significantly suppress expression of α7nAChR in mammalian cells.

### SUMMARY

The inventions described in the present disclosure are based on the finding that the suppression of α7nAChR expression by the S12 has a much more profound impact on surface α7nAChR than that in the intracellular stores, implying that S12 mainly affects receptor trafficking. The inventions described in the present disclosure are also based on the finding that the suppression effect results from S12 co-expression with α7nAChR instead of the S12 presence in extracellular milieu. The present disclosure describes inventions relating to mutant variants of a segment in the S2 segment of the S12 ectodomain, which is homologous to the hydrophobic helical motif in the α7nAChR intracellular domain, and is responsible for binding the receptor chaperone proteins RIC3 and anti-apoptotic Bcl-2 family proteins. Site-directed mutagenesis of the S2 segment abolishes the profound suppression of surface α7nAChR, suggesting that S12 competition for binding to chaperone proteins is likely an underlying mechanism leading to suppression of surface α7nAChR when S12 is co-expressed. These findings provide a new perspective for understanding certain symptoms of COVID 19 and long COVID, and for aiding in the design of potential new treatments for post-COVID syndromes.

In one aspect, the present disclosure provides a recombinant polynucleotide including a nucleic acid sequence encoding an engineered SARS-CoV-2 spike protein (S12) or an immunogenic fragment thereof, in which the engineered spike protein or immunogenic fragment includes one or more mutations in the S2 segment of the S12 ectodomain. The engineered spike protein or immunogenic fragment of the present disclosure has a reduced effect on downregulation of surface expression of α7 nicotinic acetylcholine receptor (α7nAChR) in host cells compared to a wild-type SARS-CoV-2 spike protein, or has substantially no effect on α7nAChR surface expression in host cells.

In various embodiments, the engineered spike protein or immunogenic fragment of the present disclosure includes a mutation at one or more positions selected from the group consisting of: L1145, F1148, and L1152, in which the positions correspond to a wild-type SARS-CoV-2 spike protein or orthologous sites in a variant thereof.

In various embodiments, the engineered spike protein or immunogenic fragment of the present disclosure includes mutations at two or more positions selected from the group consisting of: L1145, F1148, and L1152. In various embodiments, the engineered spike protein or immunogenic fragment of the present disclosure includes mutations at the positions of L1145, F1148, and L1152. In various embodiments, the engineered spike protein or immunogenic fragment of the present disclosure includes a mutation, which is a substitution of an alanine (A), valine (V), or glycine (G) at the one or more positions of L1145, F1148, and L1152.

In various embodiments, the engineered spike protein or immunogenic fragment of the present disclosure includes the amino acid sequence of SEQ ID NO:2, in which at least one amino acid represented by X₁, X₂ and X₃ is mutated relative to the corresponding wild-type amino acid sequence. In various embodiments, the engineered spike protein or immunogenic fragment of the present disclosure includes a sequence in which SEQ ID NO:1 is modified at the relevant amino acid sequence from 1145L to 1152L.

In various embodiments, the engineered spike protein or immunogenic fragment of the present disclosure includes the amino acid sequence selected from the group consisting of: SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, and SEQ ID NO:23. In various embodiments, the engineered spike protein or immunogenic fragment of the present disclosure includes the amino acid sequence of SEQ ID NO:6 (in which when X₁ is L, X₂ is not F, and when X₂ is F, X₁ is not L). In various embodiments, the engineered spike protein or immunogenic fragment of the present disclosure includes the amino acid sequence of SEQ ID NO:7 (in which when X₁ is L, X₂ is not L, and when X₂ is L, X₁ is not L). In various embodiments, the engineered spike protein or immunogenic fragment of the present disclosure includes the amino acid sequence of SEQ ID NO:8 (in which when X₁ is F, X₂ is not L, and when X₂ is L, X₁ is not F). In various embodiments, the engineered spike protein or immunogenic fragment of the present disclosure includes the amino acid sequence of SEQ ID NO:9, SEQ ID NO:19, SEQ ID NO:20, SEQ ID NO:21, SEQ ID NO:22, or SEQ ID NO:23. In various embodiments, the engineered spike protein or immunogenic fragment of the present disclosure further includes proline substitutions at amino acids K986 and V987 relative to the wild-type spike protein or orthologous sites in a variant thereof.

In various embodiments, the nucleic acid sequence of the present disclosure is a ribonucleic acid (RNA) selected from the group consisting of small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and any combination(s) thereof. In various embodiments, the nucleic acid is an mRNA. In various embodiments, the RNA is chemically modified with a pseudouridine. In various embodiments, the mRNA includes one or more of a stem loop, a chain terminating nucleoside, a polyA sequence, a polyadenylation signal, and/or a 5′ cap structure. In various embodiments, the engineered spike protein or immunogenic fragment thereof is encoded by a coding sequence which is codon-optimized and/or the G/C content of which is increased compared to a wild type coding sequence.

In another aspect, the present disclosure provides an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23.

In another aspect, the present disclosure provides a cell including the recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23.

In another aspect, the present disclosure provides a vaccine, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In various embodiments, the vaccine as described in the present disclosure is formulated in a nanoparticle. In various embodiments, the vaccine as described in the present disclosure is formulated in a nanoparticle is a lipid nanoparticle or a lipoplex (LPX) particle. In certain embodiments, the lipid nanoparticle includes a PEG-modified lipid, a non-cationic lipid, a sterol, an ionizable cationic lipid, or a combination thereof. In certain embodiments, the vaccine as described in the present disclosure further comprises an adjuvant.

In another aspect, the present disclosure provides a pharmaceutical formulation, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of activating an immune cell against a target antigen, the method including exposing the immune cell to an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of treating, alleviating, or managing SARS-CoV-2 infection and/or one or more symptoms thereof in a subject in need thereof, the method including administering an effective amount of a vaccine, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of treating, alleviating, or managing SARS-CoV-2 infection and/or one or more symptoms thereof in a subject in need thereof, the method including administering an effective amount of a pharmaceutical formulation, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In certain embodiments of the present disclosure, a subject is administered a single dose of the vaccine or the pharmaceutical formulation as described in the present disclosure. In certain embodiments of the present disclosure, a subject is administered an initial dose of the vaccine or the pharmaceutical formulation followed by one or more booster doses of the vaccine or the pharmaceutical formulation. In certain embodiments of the present disclosure, the vaccine or the pharmaceutical formulation is administered to the subject via intradermal injection or intramuscular injection. In various embodiments, the subject is a human. In various embodiments, the subject is at increased risk of death or serious illness from SARS-CoV-2 infection. In various embodiments, the subject is immunocompromised or has one or more comorbidities that increase the risk of death or serious illness from SARS-CoV-2 infection. In various embodiments, the subject is a pediatric subject. In various embodiments, the administration of the vaccine or the pharmaceutical formulation results in decreased side-effects relative to a comparator vaccine.

In various embodiments, the administration of the vaccine or the pharmaceutical formulation results in decreased negative effects on the nervous system and/or the immune system of the subject relative to a comparator vaccine. In various embodiments, the one or more symptoms are that of long COVID or post-COVID syndromes/conditions (PCC).

In various embodiments, the one or more symptoms are general symptoms selected from tiredness or fatigue that interferes with daily life, symptoms that get worse after physical or mental effort (also known as “post-exertional malaise”), and fever. In various embodiments, the one or more symptoms are SARS-CoV-2 and heart symptoms selected from difficulty breathing or shortness of breath, cough, chest pain, and fast-beating or pounding heart (also known as heart palpitations). In various embodiments, the one or more symptoms are neurological symptoms selected from difficulty thinking or concentrating (sometimes referred to as “brain fog”), headache, sleep problems, dizziness when you stand up (lightheadedness), pins-and-needles feelings, change in smell or taste, and depression or anxiety. In various embodiments, the one or more symptoms are digestive symptoms selected from diarrhea or stomach pain. In various embodiments, the one or more symptoms are selected from Joint or muscle pain, rash, and changes in menstrual cycles.

### BRIEF DESCRIPTION OF THE DRAWINGS

FIG. 1 depicts S12 expression downregulates native surface α7nAChR in PC12 cells. FIG. 1 is a bar graph showing normalized mean relative intensities of anti-α7nAChR staining showing ˜37% reduction of α7nAChR labeling in the +S12 group compared to the control. Data for each group were collected from 3 independent experiments with a total n=4689 (control) and n=3293 (+S12) cells. Data are means±SEM; p≤0.0001 (****) from two- tailed unpaired t test.

FIGS. 2A-2B are data showing co-expression of S12 downregulates surface α7nAChR in HEK293T/17 cells. FIG. 2A is a bar graph showing relative mean intensities of anti-α7nAChR staining show ˜35% reduction of surface α7nAChR in the +S12 group compared to the control. Data for each group were from 3 independent experiments with a total n=9010 (control) and n=7636 (+S12) cells. FIG. 2B is a bar graph Relative mean intensities of α-BTX staining show ˜57% reduction of functional α7nAChR in the +S12 group compared to the control. Data for each group were collected from two independent experiments with a total n=3384 (control) and n=1587 (+S12) cells. All data are means±SEM; p≤0.0001(****) from two-tailed unpaired t test.

FIG. 3 is a bar graph showing co-expression of S12 with α4β2nAChR did not affect surface α4β2 expression. Quantified relative mean intensities of anti-β2nAChR staining show no significant difference in surface β2nAChR labeling between the +S12 and control groups. Data for each group were from 2 independent experiments with a total number of cells n=1537 (control) and n=1289 (+S12). Data are means ±SEM; p>0.05 (ns) from two-tailed unpaired t test.

FIGS. 4A-4B are bar graphs showing that S12 in the media does not suppress surface α7nAChR expression. FIG. 4A is a bar graph showing relative mean intensities of anti-α7nAChR labeling of non-permeabilized HEK293T/17 cells transiently co-transfected with cDNAs encoding α7nAChR, GFP, RIC3 and NACHO. Media was shared with cells in the well inserts transiently transfected with cDNA encoding GFP (control) or cotransfected with GFP+S12 (S12 media) for 5 days. No significant difference was observed between groups. Data for each group was from 2 independent experiments with a total number of cells n=21066 (control) and n=20677 (S12 media). FIG. 4B is a bar graph showing relative mean intensities of a-bungarotoxin (α-BTX) labeling of functional α7nAChR from the same groups as in (b) with a total of n=5592 (control) and n=4871 (S12 media) cells. All data are means±SEM; p>0.05 (ns) from two-tailed unpaired t test.

FIG. 5 is a bar graph showing relative mean intensities of anti-α7nAChR staining show ˜9% reduction of α7nAChR labeling in the +S12 group compared to the control. Data for each group were from 2 independent experiments with a total n=8867 (control) and n=6655 (+S12) cells. All data are presented as means±SEM; p≤0.0001(****) from two-tailed unpaired t test.

FIG. 6A depicts a helical segment of S12 (1145L-1152L) homologous to the intracellular chaperone-binding motif of α7nAChR (L411-V418) is primarily responsible for the S12-induced downregulation of surface α7nAChR. Structures of α7nAChR transmembrane and intracellular domains (PDB: 7RPM) and S12 (PDB: 7VXF) showing the helical motif known for binding α7nAChR chaperones. Sequence alignments of the helical segments highlighting three hydrophobic residues (bold) critical for binding α7nAChR chaperones and their mutations in S12_{AAA}. FIG. 6A discloses SEQ ID NO:s 10, 25 and 9, respectively, in order of appearance.

FIG. 6B is a bar graph showing relative mean intensities of anti-α7nAChR immunofluorescent staining of non- permeabilized HEK293 cells showing that relative to the control group (α7nAChR- GFP/RIC3/NACHO), co-expressing S12_{AAA} (+S12_{AAA}) caused only ˜5% reduction of surface α7nAChR in contrast to ˜35% reduction by S12 co-expression (+S12, adapted from FIG. 2). Data for each group were from 2 independent experiments with a total number of cells n=8594 (control), 4903 (+S12_{AAA}).

FIG. 6C is a bar graph showing relative mean intensities of anti-α7nAChR immunofluorescent staining of non-permeabilized PC12 cells transiently transfected with cDNA of GFP alone (control) or +S12_{AAA}. In contrast to ˜37% reduction by S12 co-expression (red, adapted from FIG. 1), +S12_{AAA} co-expression (blue) produced no significant difference in the staining intensity compared to the control. Data for each group were from 3 independent experiments with a total number of cells n=7853 (control) and n=5614 (+S12_{AAA}). Data are means±SEM; p values are from one-way ANOVA with Dunnett's multiple comparisons; p>0.05 (ns), p≤0.01 (**), p≤0.0001(****).

FIG. 6D is an immunoblot showing pulldown of RIC3 by S12 or S12_{AAA} from lysates of HEK293 cells coexpressing RIC3 and S12 or S12_{AAA}. The His-tags on the S12 constructs were used for pulldowns using NiNTA resin. The bound proteins were probed by anti-RIC3 (1:100) and anti-SARS-CoV-2 spike protein (1:250).

FIG. 7 is a graph showing a time course of apoptosis in differentiated PC12 cells transfected with cDNAs encoding mCerulean (control, black triangles) or mCerulean plus either S12 (squares) or S12_{AAA} (circles). Apoptosis was induced by serum deprivation and 1 μM staurosporine and measured as the percentage of transfected cells with activated caspase-3/7 activity. The measurements were performed ˜4 days after transfections. Data for each group were from three independent experiments with a total number of cells n=3076 (control), n=2170 (+S12_{AAA}), and n=3085 (+S12).

### DETAILED DESCRIPTION

To facilitate an understanding of the present invention, a number of terms and phrases are defined below.

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., rodents, simians, equines, bovines, porcines, canines, felines, and the like), and more preferably include humans.

As used herein, the term “effective amount” refers to the amount of a recombinant nucleic acid, protein, polypeptide, or a vaccine or a pharmaceutical composition/formulation of the present invention sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof. In some embodiments, an “effective amount” of a recombinant nucleic acid, protein, polypeptide, or a vaccine or a pharmaceutical composition/formulation described herein, stimulates an immune response in the subject.

The terms “treat,” “treating,” or “treatment,” and other grammatical equivalents as used in this disclosure, include alleviating, abating, ameliorating, or preventing a disease, e.g., a viral infection, and related condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic preventive benefit. By therapeutic benefit is meant eradication or amelioration of the underlying disorder being treated. “Prevent” or “prophylaxis” as used herein includes modulating the immune response of a subject, such that immunization against the viral infection results. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

As used herein, the term “vaccine” (also known in the art as “vaccination”) refers to an immunogenic composition comprising a biological agent, e.g., a recombinant nucleic acid, protein, polypeptide, which elicits an immune response to a specific antigen from an infectious disease-causing pathogen, e.g., a protein fragment from a virus, e.g., a coronavirus, to enhance immunity against said infectious disease-causing pathogen. In some embodiments administration of a vaccine, e.g., an effective amount of a vaccine, may induce a cytotoxic T-cell response and/or a helper T-cell response. In some embodiments administration of a vaccine, e.g., an effective amount of a vaccine, may induce a B-cell response, e.g., production of antibodies.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular recombinant nucleic acid, protein, or polypeptide, that recombinant nucleic acid, protein, or polypeptide can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the invention unless claimed. No language in the specification should be construed as indicating any non-claimed element as essential to the practice of the present disclosure.

A deficiency of the functional α7 nicotinic acetylcholine receptor (α7nAChR) impairs neuronal and immune systems. The SARS-CoV-2 spike protein (S12) facilitates virus cell entry during COVID-19 infection and can also independently disrupt cellular functions. The invention described in the present disclosure relates to the findings that S12 expression significantly downregulates surface expression of α7nAChR in mammalian cells. The inventions relate to the discovery that a helical segment of S12 (L1145-L1152) is responsible for the downregulation of expression of α7nAChR, as the mutant S12_{AAA} (L1145A-F1148A-L1152A) has minimal effects on surface α7nAChR expression. This S12 segment is homologous to the α7nAChR intracellular helical motif known to bind RIC3 and anti-apoptotic Bcl-2 family proteins to promote α7nAChR surface expression. Competition from S12 for binding these proteins may underly suppression of surface α7nAChR. Considering the critical roles of α7nAChR in cellular functions, the present disclosure provides improved mRNA vaccines and treatment options for certain symptoms related to COVID-19 and long COVID.

In one aspect, the present disclosure provides a recombinant polynucleotide including a nucleic acid sequence encoding an engineered SARS-CoV-2 spike protein (S12) or an immunogenic fragment thereof, in which the engineered spike protein or immunogenic fragment includes one or more mutations in the S2 segment of the S12 ectodomain. The engineered spike protein or immunogenic fragment of the present disclosure has a reduced effect on downregulation of surface expression of α7 nicotinic acetylcholine receptor (α7nAChR) in host cells compared to a wild-type SARS-CoV-2 spike protein, or has substantially no effect on α7nAChR surface expression in host cells.

In various embodiments, the engineered spike protein or immunogenic fragment of the present disclosure includes a mutation at one or more positions selected from the group consisting of: L1145, F1148, and L1152, in which the positions correspond to a wild-type SARS-CoV-2 spike protein or orthologous sites in a variant thereof.

In various embodiments, the engineered spike protein or immunogenic fragment of the present disclosure includes mutations at two or more positions selected from the group consisting of: L1145, F1148, and L1152. In various embodiments, the engineered spike protein or immunogenic fragment of the present disclosure includes mutations at the positions of L1145, F1148, and L1152. In various embodiments, the engineered spike protein or immunogenic fragment of the present disclosure includes a mutation, which is a substitution of an alanine (A), valine (V), or glycine (G) at the one or more positions of L1145, F1148, and L1152.

In various embodiments, the engineered spike protein or immunogenic fragment of the present disclosure includes the amino acid sequence of SEQ ID NO:2, in which at least one amino acid represented by X₁, X₂ and X₃ is mutated relative to the corresponding wild-type amino acid sequence. In various embodiments, the engineered spike protein or immunogenic fragment of the present disclosure includes a sequence in which SEQ ID NO:1 is modified at the relevant amino acid sequence from 1145L to 1152L.

In various embodiments, the engineered spike protein or immunogenic fragment of the present disclosure includes the amino acid sequence selected from the group consisting of: SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, and SEQ ID NO:23. In various embodiments, the engineered spike protein or immunogenic fragment of the present disclosure includes the amino acid sequence of SEQ ID NO:6 (in which when X₁ is L, X₂ is not F, and when X₂ is F, X₁ is not L). In various embodiments, the engineered spike protein or immunogenic fragment of the present disclosure includes the amino acid sequence of SEQ ID NO:7 (in which when X₁ is L, X₂ is not L, and when X₂ is L, X₁ is not L). In various embodiments, the engineered spike protein or immunogenic fragment of the present disclosure includes the amino acid sequence of SEQ ID NO:8 (in which when X₁ is F, X₂ is not L, and when X₂ is L, X₁ is not F). In various embodiments, the engineered spike protein or immunogenic fragment of the present disclosure includes the amino acid sequence of SEQ ID NO:9, SEQ ID NO:19, SEQ ID NO:20, SEQ ID NO:21, SEQ ID NO:22, or SEQ ID NO:23. In various embodiments, the engineered spike protein or immunogenic fragment of the present disclosure further includes proline substitutions at amino acids K986 and V987 relative to the wild-type spike protein or orthologous sites in a variant thereof.

In various embodiments, the nucleic acid sequence of the present disclosure is a ribonucleic acid (RNA) selected from the group consisting of small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and any combination(s) thereof. In various embodiments, the nucleic acid is an mRNA. In various embodiments, the RNA is chemically modified with a pseudouridine. In various embodiments, the mRNA includes one or more of a stem loop, a chain terminating nucleoside, a polyA sequence, a polyadenylation signal, and/or a 5′ cap structure. In various embodiments, the engineered spike protein or immunogenic fragment thereof is encoded by a coding sequence which is codon-optimized and/or the G/C content of which is increased compared to a wild type coding sequence.

In another aspect, the present disclosure provides an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23.

In another aspect, the present disclosure provides a cell including the recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23.

In another aspect, the present disclosure provides a vaccine, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In various embodiments, the vaccine as described in the present disclosure is formulated in a nanoparticle. In various embodiments, the vaccine as described in the present disclosure is formulated in a nanoparticle is a lipid nanoparticle or a lipoplex (LPX) particle. In certain embodiments, the lipid nanoparticle includes a PEG-modified lipid, a non-cationic lipid, a sterol, an ionizable cationic lipid, or a combination thereof. In certain embodiments, the vaccine as described in the present disclosure further comprises an adjuvant.

In another aspect, the present disclosure provides a pharmaceutical formulation, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of activating an immune cell against a target antigen, the method including exposing the immune cell to an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of treating, alleviating, or managing SARS-CoV-2 infection and/or one or more symptoms thereof in a subject in need thereof, the method including administering an effective amount of a vaccine, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

In another aspect, the present disclosure provides a method of treating, alleviating, or managing SARS-CoV-2 infection and/or one or more symptoms thereof in a subject in need thereof, the method including administering an effective amount of a pharmaceutical formulation, which includes a recombinant polynucleotide encoding an engineered spike protein or immunogenic fragment that includes a sequence of SEQ ID NO:3, SEQ ID NO:4, SEQ ID NO:5, SEQ ID NO:6 (in which when X₁ is L, X₂ is not F, and when X₂ is F, X₁ is not L), SEQ ID NO:7 (in which when X₁ is L, X₂ is not L, and when X₂ is L, X₁ is not L), SEQ ID NO:8 (in which when X₁ is F, X₂ is not L, and when X₂ is L, X₁ is not F), SEQ ID NO:9, SEQ ID NO:19, SEQ ID NO:20, SEQ ID NO:21, SEQ ID NO:22, or SEQ ID NO:23; and a pharmaceutically acceptable carrier.

### Chemical Modifications

Vaccines of the present disclosure, in some embodiments, comprise at least RNA (e.g. mRNA) polynucleotide having an open reading frame encoding at least one antigenic polypeptide that comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleotide linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the vaccines of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladeno sine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′, 2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′, 2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′ azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′, 2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2- [2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene;2 (amino)purine;2,4,5-(trimethyl)phenyl; 2′ methyl, 2′amino, 2′azido, 2′fluro-cytidine;2′ methyl, 2′ amino, 2′azido, 2′fluro-adenine;2′methyl, 2′amino, 2′ azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo) -phenoxazin- 1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m.sup.1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m^{1ψ}), 5-methoxy-uridine (mo^{5U}), 5-methyl-cytidine (m^{5C}), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (v) and 5-methyl-cytidine (m^{5C}). In some embodiments, polynucleotides (e.g., RNA polynucleotides, such as mRNA polynucleotides) comprise 1-methyl-pseudouridine (m^{1ψ}). In some embodiments, polynucleotides (e.g., RNA polynucleotides, such as mRNA polynucleotides) comprise 1-methyl-pseudouridine (m^{1ψ}) and 5-methyl-cytidine (m^{5C}). In some embodiments, polynucleotides (e.g., RNA polynucleotides, such as mRNA polynucleotides) comprise 2-thiouridine (s^{2U}). In some embodiments, polynucleotides (e.g., RNA polynucleotides, such as mRNA polynucleotides) comprise 2-thiouridine and 5-methyl-cytidine (m^{5C}). In some embodiments, polynucleotides (e.g., RNA polynucleotides, such as mRNA polynucleotides) comprise methoxy-uridine (mo^{5U}). In some embodiments, polynucleotides (e.g., RNA polynucleotides, such as mRNA polynucleotides) comprise 5-methoxy-uridine (mo^{5U}) and 5-methyl-cytidine (m^{5C}). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m^{5C}). In some embodiments, polynucleotides (e.g., RNA polynucleotides, such as mRNA polynucleotides) comprise N6-methyl-adenosine (m^{6A}). In some embodiments, polynucleotides (e.g., RNA polynucleotides, such as mRNA polynucleotides) comprise N6-methyl-adenosine (m^{6A}) and 5-methyl-cytidine (m^{5C}).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m^{5C}), meaning that all cytosine residues in the mRNA sequence are replaced with 5-methyl-cytidine (m^{5C}). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m^{5C}), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). n some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Thus, in some embodiments, the RNA (e.g., mRNA) vaccines comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s^{2U}), 4-thio-uridine (s^{4U}), 4-thio-pseudouridine, 2-thio-pseudouridine, 5-hydroxy-uridine (ho^{5U}), 5-aminoallyl-uridine, 5-halo-uridine (e.g., 5-iodo-uridineor 5-bromo-uridine), 3-methyl-uridine (m^{3U}), 5-methoxy-uridine (mo^{5U}), uridine 5-oxyacetic acid (cmo^{5U}), uridine 5-oxyacetic acid methyl ester (mcmo^{5U}), 5-carboxymethyl-uridine (cm^{5U}), 1-carboxymethyl-pseudouridine, 5-carboxyhydroxymethyl-uridine (chm^{5U}), 5-carboxyhydroxymethyl-uridine methyl ester (mchm^{5U}), 5-methoxycarbonylmethyl-uridine (mcm^{5U}), 5-methoxycarbonylmethyl-2-thio-uridine (mcm.sup.5s.sup.2U), 5-aminomethyl-2-thio-uridine (nm.sup.5s.sup.2U), 5-methylaminomethyl-uridine (mnm.sup.5U), 5-methylaminomethyl-2-thio-uridine (mnm.sup.5s.sup.2U), 5-methylaminomethyl-2-seleno-uridine (mnm.sup.5se.sup.2U), 5-carbamoylmethyl-uridine (ncm.sup.5U), 5-carboxymethylaminomethyl-uridine (cmnm.sup.5U), 5-carboxymethylaminomethyl-2-thio-uridine (cmnm^{5s2U}), 5-propynyl-uridine, 1-propynyl-pseudouridine, 5-taurinomethyl-uridine (τm^{5U}), 1-taurinomethyl-pseudouridine, 5-taurinomethyl-2-thio-uridine(m.sup.5s.sup.2U), 1-taurinomethyl-4-thio-pseudouridine, 5-methyl-uridine (m.sup.5U, i.e., having the nucleobase deoxythymine), 1-methyl-pseudouridine (m.sup.1ψ), 5-methyl-2-thio-uridine (m5s.sup.2U), 1-methyl-4-thio-pseudouridine (m.sup.1s.sup.4ψ), 4-thio-1-methyl-pseudouridine, 3-methyl-pseudouridine (m.sup.3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m.sup.5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp.sup.3U), 1-methyl-3-(3-amino-3-carboxypropyl)pseudouridine (acp.sup.3ψ), 5-(isopentenylaminomethyl)uridine (inm.sup.5U), 5-(isopentenylaminomethyl)-2-thio-uridine (inm.sup.5s.sup.2U), α-thio-uridine, 2′-O-methyl-uridine (Um), 5,2′-O-dimethyl-uridine (msUm), 2′-O-methyl-pseudouridine (Wm), 2-thio-2′-O-methyl-uridine (s.sup.2Um), 5-methoxycarbonylmethyl-2′-O-methyl-uridine (mcm.sup.5Um), 5-carbamoylmethyl-2′-O-methyl-uridine (ncm.sup.5Um), 5-carboxymethylaminomethyl-2′-O-methyl-uridine (cmnm.sup.5Um), 3,2′-O-dimethyl-uridine (m.sup.3Um), and 5-(isopentenylaminomethyl)-2′-O-methyl-uridine (inm.sup.5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m.sup.3C), N4-acetyl-cytidine (ac.sup.4C), 5-formyl-cytidine (f.sup.5C), N4-methyl-cytidine (m.sup.4C), 5-methyl-cytidine (m.sup.5C), 5-halo-cytidine (e.g., 5-iodo-cytidine), 5-hydroxymethyl-cytidine (hm.sup.5C), 1-methyl-pseudoisocytidine, pyrrolo-cytidine, pyrrolo-pseudoisocytidine, 2-thio-cytidine (s.sup.2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k.sub.2C), α-thio-cytidine, 2′-O-methyl-cytidine (Cm), 5,2′-O-dimethyl-cytidine (m.sup.5Cm), N4-acetyl-2′-O-methyl-cytidine (ac.sup.4Cm), N4,2′-O-dimethyl-cytidine (m.sup.4Cm), 5-formyl-2′-O-methyl-cytidine (f.sup.5Cm), N4,N4,2′-O-trimethyl-cytidine (m.sup.42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m.sup.1A), 2-methyl-adenine (m.sup.2A), N6-methyl-adenosine (m.sup.6A), 2-methylthio-N6-methyl-adenosine (ms.sup.2m.sup.6A), N6-isopentenyl-adenosine (i.sup.6A), 2-methylthio-N6-isopentenyl-adenosine (ms.sup.2i.sup.6A), N6-(cis-hydroxyisopentenyl)adenosine (io.sup.6A), 2-methylthio-N6-(cis-hydroxyisopentenyl)adenosine (ms.sup.2io.sup.6A), N6-glycinylcarbamoyl-adenosine (g.sup.6A), N6-threonylcarbamoyl-adenosine (t.sup.6A), N6-methyl-N6-threonylcarbamoyl-adenosine (m.sup.6t6A), 2-methylthio-N6-threonylcarbamoyl-adenosine (ms.sup.2g.sup.6A), N6,N6-dimethyl-adenosine (m.sup.62A), N6-hydroxynorvalylcarbamoyl-adenosine (hn.sup.6A), 2-methylthio-N6-hydroxynorvalylcarbamoyl-adenosine (ms.sup.2hn.sup.6A), N6-acetyl-adenosine (ac.sup.6A), 7-methyl-adenine, 2-methylthio-adenine, 2-methoxy-adenine, α-thio-adenosine, 2′-O-methyl-adenosine (Am), N6,2′-O-dimethyl-adenosine (m.sup.6Am), N6,N6,2′-O-trimethyl-adenosine (m.sup.62Am), 1,2′-O-dimethyl-adenosine (m.sup.1Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m.sup.1I), wyosine (imG), methylwyosine (mimG), 4-demethyl-wyosine (imG-14), isowyosine (imG2), wybutosine (yW), peroxywybutosine (o.sub.2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ.sub.0), 7-aminomethyl-7-deaza-guanosine (preQ.sub.1), archaeosine (G.sup.+), 7-deaza-8-aza-guanosine, 6-thio-guanosine, 6-thio-7-deaza-guanosine, 6-thio-7-deaza-8-aza-guanosine, 7-methyl-guanosine (m.sup.7G), 6-thio-7-methyl-guanosine, 7-methyl-inosine, 6-methoxy-guanosine, 1-methyl-guanosine (mG), N2-methyl-guanosine (m.sup.2G), N2,N2-dimethyl-guanosine (m.sup.22G), N2,7-dimethyl-guanosine (m.sup.2,7G), N2, N2,7-dimethyl-guanosine (m.sup.2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m.sup.2Gm), N2,N2-dimethyl-2′-O-methyl-guanosine (m.sup.22Gm), 1-methyl-2′-O-methyl-guanosine (mGm), N2,7-dimethyl-2′-O-methyl-guanosine (m.sup.2′7Gm), 2′-O-methyl-inosine (Im), 1,2′-O-dimethyl-inosine (m.sup.1Im), 2′-O-ribosylguanosine (phosphate) (Gr(p)), 1-thio-guanosine, 06-methyl-guanosine, 2′-F-ara-guanosine, and 2′-F-guanosine.

### Stabilizing Elements

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA (e.g., mRNA) vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA (e.g., mRNA) vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA (e.g., mRNA) vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA (e.g., mRNA) vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, including (e.g., consisting of) a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA (e.g., mRNA) vaccine may have one or more AU-rich sequences removed. These sequences, sometimes referred to as AURES are destabilizing sequences found in the 3′UTR. The AURES may be removed from the RNA (e.g., mRNA) vaccines. Alternatively the AURES may remain in the RNA (e.g., mRNA) vaccine.

### Nanoparticle Formulations

In some embodiments, SARS-CoV-2 virus RNA (e.g. mRNA) vaccines are formulated in a nanoparticle. In some embodiments, SARS-CoV-2 virus RNA (e.g. mRNA) vaccines are formulated in a lipid nanoparticle. In some embodiments, SARS-CoV-2 virus RNA (e.g. mRNA) vaccines are formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, SARS-CoV-2 virus RNA (e.g., mRNA) vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a SARS-CoV-2 virus RNA (e.g. mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids.

The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z, 12Z)-octadeca-9,12-dien- 1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In some embodiments, lipid nanoparticle formulations include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 38.5% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 15% of the neutral lipid, 40% of the sterol, and 5% of the PEG or PEG-modified lipid on a molar basis.

In some embodiments, lipid nanoparticle formulations include 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), 7.5% of the neutral lipid, 31.5% of the sterol, and 3.5% of the PEG or PEG-modified lipid on a molar basis.

In some embodiments, lipid nanoparticle formulations consists essentially of a lipid mixture in molar ratios of 20-70% cationic lipid: 5-45% neutral lipid: 20-55% cholesterol: 0.5-15% PEG-modified lipid. In some embodiments, lipid nanoparticle formulations consists essentially of a lipid mixture in a molar ratio of 20-60% cationic lipid: 5-25% neutral lipid: 25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DS G), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the SARS-CoV-2 virus RNA (e.g. mRNA) vaccine composition may comprise the polynucleotide described herein, formulated in a lipid nanoparticle comprising MC3, Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 hMPV), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

### Liposomes, Lipoplexes, and Lipid Nanoparticles

The RNA (e.g., mRNA) vaccines of the disclosure can be formulated using one or more liposomes, lipoplexes, or lipid nanoparticles. In some embodiments, pharmaceutical compositions of RNA (e.g., mRNA) vaccines include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]--dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, the RNA (e.g., mRNA) vaccine pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the cationic lipid may be a low molecular weight cationic lipid such as those described in U.S. Patent Application No. 20130090372, the contents of which are herein incorporated by reference in their entirety.

In some embodiments, the RNA (e.g., mRNA) vaccines may be formulated in a lipid vesicle, which may have crosslinks between functionalized lipid bilayers.

In some embodiments, the RNA (e.g., mRNA) vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the RNA (e.g., mRNA) vaccines may be formulated in a lipid-polycation complex, which may further include a non-cationic lipid such as, but not limited to, cholesterol or dioleoyl phosphatidylethanolamine (DOPE).

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the RNA (e.g., mRNA) vaccines may be formulated in a lipid nanoparticle.

In some embodiments, the RNA (e.g., mRNA) vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z, 12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z, 12Z)-octadeca-9,12-dien-1-yloxy]-2-[(octyloxy)methyl]propan-1-ol (Compound 3 in US20130150625); and 2-(dimethylamino)-3-[(9Z, 12Z)-octadeca-9,12-dien-1-yloxy]-2-{[(9Z, 12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, the lipid nanoparticle formulation consists essentially of (i) at least one lipid selected from the group consisting of 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the formulation includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety)

In some embodiments, the formulations of the present disclosure include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, the formulations of the present disclosure include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, the formulations of the present disclosure include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, the formulations of the present disclosure include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In some embodiments, the formulations of the present disclosure include about 40% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In some embodiments, the formulations of the present disclosure include about 57.2% of a cationic lipid selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In some embodiments, the formulations of the present disclosure include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), about 7.5% of the neutral lipid, about 31.5% of the sterol, and about 3.5% of the PEG or PEG-modified lipid on a molar basis.

In some embodiments, lipid nanoparticle formulation consists essentially of a lipid mixture in molar ratios of about 20-70% cationic lipid: 5-45% neutral lipid: 20-55% cholesterol: 0.5-15% PEG-modified lipid; more preferably in a molar ratio of about 20-60% cationic lipid: 5-25% neutral lipid: 25-55% cholesterol: 0.5-15% PEG-modified lipid.

In some embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Examples of lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16, 19-dien-8-amine, (13Z,16Z)-N,N-dimethyldocosa-13,16-dien-5-amine, (12Z, 15Z)-N,N-dimethylhenicosa-12,15-dien-4-amine, (14Z, 17Z)-N,N-dimethyltricosa-14,17-dien-6-amine, (15Z, 18Z)-N,N-dimethyltetracosa-15,18-dien-7-amine, (18Z,21Z)-N,N-dimethylheptacosa-18,21-dien-10-amine, (15Z, 18Z)-N,N-dimethyltetracosa-15,18-dien-5-amine, (14Z, 17Z)-N,N-dimethyltricosa-14,17-dien-4-amine, (19Z,22Z)-N, N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z, 19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyleptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos -20-en-10-amine, (22Z)-N,N-dimethylhentriacont-22-en-10-amine, (16Z)-N,N-dimethylpentacos-16-en-8-amine, (12Z, 15Z)-N,N-dimethyl-2-nonylhenicosa-12,15-dien-1-amine, (13Z, 16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3 -{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z, 12Z)-octadeca-9,12-dien-1-yloxy]-3-(octyloxy)propan-2-amine, S-N,N-dimethyl-1-[(9Z, 12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z, 12Z)-octadeca-9,12-dien-1-yloxy]-3-[(5Z)-oct-5-en-1-yloxy]propan-2-amine, 1-{2-[(9Z, 12Z)-octadeca-9,12-dien-1-yloxy]-1-[(octyloxy)methyl]ethyl}azetidine, (2S)-1-(hexyloxy)-N,N-dimethyl-3-[(9Z, 12Z)-octadeca-9,12-dien-1-yloxy]propan-2-amine, (2S)-1-(heptyloxy)-N,N-dimethyl-3-[(9Z, 12Z)-octadeca-9,12-dien-1-yloxy]propan-2-amine, N,N-dimethyl-1-(nonyloxy)-3-[(9Z, 12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z) -icosa-11,14-dien-1-yloxyl-N,N-dimethyl-3-(pentyloxy)propan-2-amine, (2S)-1-(hexyloxy)-3-[(11Z,14Z)-icosa-11,14-dien- 1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z, 16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1- [(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoylo ctyl)oxy]-3-[(9Z, 12Z)-octadeca-9,12-dien-1-yloxy]propan-2-amine, (2R)-1-[(3,7-dimethyloctyl)oxy]-N,N-dimethyl-3-[(9Z, 12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the LNP formulations of the RNA (e.g., mRNA) vaccines may contain PEG-c-DOMG at 3% lipid molar ratio. In some embodiments, the LNP formulations of the RNA (e.g., mRNA) vaccines may contain PEG-c-DOMG at 1.5% lipid molar ratio.

In some embodiments, the pharmaceutical compositions of the RNA (e.g., mRNA) vaccines may include at least one of the PEGylated lipids described in International Publication No. WO2012099755, the contents of which are herein incorporated by reference in their entirety.

In some embodiments, the LNP formulation may contain PEG-DMG 2000 (1,2-dimyristoyl-sn-glycero-3-phophoethanolamine-N-[methoxy(polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA (e.g., mRNA) vaccines, PNAS 2012; PMID: 22908294, the contents of each of which are herein incorporated by reference in their entirety).

The lipid nanoparticles described herein may be made in a sterile environment.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013033438, the contents of which are herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In some embodiments, polymer conjugates with the polynucleotides of the present disclosure may be made using the methods and/or segmented polymeric reagents described in U.S. Patent Application No. 20130072709, the contents of which are herein incorporated by reference in its entirety. In some embodiments, the polymer conjugate may have pendant side groups comprising ring moieties such as, but not limited to, the polymer conjugates described in U.S. Patent Publication No. US20130196948, the contents which are herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject.

In some embodiments, the RNA (e.g., mRNA) vaccines of the present disclosure are formulated in nanoparticles which comprise a conjugate to enhance the delivery of the nanoparticles of the present disclosure in a subject.

In some embodiments, RNA (e.g., mRNA) vaccine pharmaceutical compositions comprising the polynucleotides of the present disclosure and a conjugate that may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Patent Publication No. US20130184443, the contents of which are herein incorporated by reference in their entirety.

The nanoparticle formulations may be a carbohydrate nanoparticle comprising a carbohydrate carrier and a RNA (e.g., mRNA) vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in their entirety).

Nanoparticle formulations of the present disclosure may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA (e.g., mRNA) vaccines within the central nervous system. As a non-limiting example nanoparticles comprising a hydrophilic coating and methods of making such nanoparticles are described in U.S. Patent Publication No. US20130183244, the contents of which are herein incorporated by reference in their entirety.

In some embodiments, the lipid nanoparticles of the present disclosure may be hydrophilic polymer particles. Non-limiting examples of hydrophilic polymer particles and methods of making hydrophilic polymer particles are described in U.S. Patent Publication No. US20130210991, the contents of which are herein incorporated by reference in their entirety.

In some embodiments, the lipid nanoparticles of the present disclosure may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbon.

In some embodiments, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, SARS-CoV-2 (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e., a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in their entirety. The polymeric material may additionally be irradiated. As a non-limiting example, the polymeric material may be gamma irradiated (see e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, poly siloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., U.S. Publication 20120121718 and U.S. Publication 20100003337 and U.S. Pat. No. 8,263,665, the contents of each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in their entirety). A non-limiting scalable method to produce nanoparticles which can penetrate human mucus is described by Xu et al. (see, e.g., J Control Release 2013, 170(2):279-86; the contents of which are herein incorporated by reference in their entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin 34 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., U.S. Publication 20100215580 and U.S. Publication 20080166414 and US20130164343; the contents of each of which are herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion, which may increase the delivery of the mucus penetrating lipid nanoparticles to the mucosal tissue.

In some embodiments, the mucus penetrating lipid nanoparticles may be a hypotonic formulation comprising a mucosal penetration enhancing coating. The formulation may be hypotonic for the epithelium to which it is being delivered. Non-limiting examples of hypotonic formulations may be found in International Patent Publication No. WO2013110028, the contents of which are herein incorporated by reference in their entirety.

In some embodiments, in order to enhance the delivery through the mucosal barrier the RNA (e.g., mRNA) vaccine formulation may comprise or be a hypotonic solution.

Hypotonic solutions were found to increase the rate at which mucoinert particles such as, but not limited to, mucus-penetrating particles, were able to reach the vaginal epithelial surface (see e.g., Ensign et al. Biomaterials 2013 34(28):6922-9, the contents of which are herein incorporated by reference in their entirety).

In some embodiments, the RNA (e.g., mRNA) vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132, the contents of each of which are incorporated herein by reference in their entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, the contents of each of which are incorporated herein by reference in their entirety). One example of passive targeting of formulations to liver cells includes the DLin-DMA, DLin-KC2-DMA and DLin-MC3-DMA-based lipid nanoparticle formulations, which have been shown to bind to apolipoprotein E and promote binding and uptake of these formulations into hepatocytes in vivo (Akinc et al. Mol Ther. 2010 18:1357-1364, the contents of which are incorporated herein by reference in their entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, the contents of each of which are incorporated herein by reference in their entirety).

In some embodiments, the RNA (e.g., mRNA) vaccine is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2 (8), pp 1696-1702; the contents of which are herein incorporated by reference in their entirety). As a non-limiting example, the SLN may be the SLN described in International Patent Publication No. WO2013105101, the contents of which are herein incorporated by reference in their entirety. As another non-limiting example, the SLN may be made by the methods or processes described in International Patent Publication No. WO2013105101, the contents of which are herein incorporated by reference in their entirety.

Liposomes, lipoplexes, or lipid nanoparticles may be used to improve the efficacy of polynucleotides directed protein production as these formulations may be able to increase cell transfection by the RNA (e.g., mRNA) vaccine; and/or increase the translation of encoded protein. One such example involves the use of lipid encapsulation to enable the effective systemic delivery of polyplex plasmid DNA (Heyes et al., Mol Ther. 2007 15:713-720; the contents of which are incorporated herein by reference in their entirety). The liposomes, lipoplexes, or lipid nanoparticles may also be used to increase the stability of the polynucleotide.

In some embodiments, the RNA (e.g., mRNA) vaccines of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the RNA (e.g., mRNA) vaccines may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106, the contents of each of which are incorporated herein by reference in their entirety).

In some embodiments, the RNA (e.g., mRNA) vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA (e.g., mRNA) vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA (e.g., mRNA) vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the RNA (e.g., mRNA) vaccine controlled release and/or targeted delivery formulation comprising at least one polynucleotide may comprise at least one PEG and/or PEG related polymer derivatives as described in U.S. Pat. No. 8,404,222, the contents of which are incorporated herein by reference in their entirety.

In some embodiments, the RNA (e.g., mRNA) vaccine controlled release delivery formulation comprising at least one polynucleotide may be the controlled release polymer system described in US20130130348, the contents of which are incorporated herein by reference in their entirety.

In some embodiments, the RNA (e.g., mRNA) vaccines of the present disclosure may be encapsulated in a therapeutic nanoparticle, referred to herein as “therapeutic nanoparticle RNA (e.g., mRNA) vaccines.” Therapeutic nanoparticles may be formulated by methods described herein and known in the art such as, but not limited to, International Pub Nos. WO2010005740, WO2010030763, WO2010005721, WO2010005723, WO2012054923, U.S. Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In some embodiments, therapeutic polymer nanoparticles may be identified by the methods described in US Pub No. US20120140790, the contents of which are herein incorporated by reference in their entirety.

In some embodiments, the therapeutic nanoparticle RNA (e.g., mRNA) vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present disclosure (see International Pub No. 2010075072 and US Pub No. US20100216804, US20110217377 and US20120201859, the contents of each of which are incorporated herein by reference in their entirety). In another non-limiting example, the sustained release formulation may comprise agents which permit persistent bioavailability such as, but not limited to, crystals, macromolecular gels and/or particulate suspensions (see U.S. Patent Publication No US20130150295, the contents of each of which are incorporated herein by reference in their entirety).

In some embodiments, the therapeutic nanoparticle RNA (e.g., mRNA) vaccines may be formulated to be target specific. As a non-limiting example, the therapeutic nanoparticles may include a corticosteroid (see International Pub. No. WO2011084518, the contents of which are incorporated herein by reference in their entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, the contents of each of which are incorporated herein by reference in their entirety.

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the therapeutic nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052, the contents of which are incorporated herein by reference in their entirety.

As a non-limiting example the therapeutic nanoparticle comprises a PLGA-PEG block copolymer (see U.S. Publication No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in their entirety). In yet another non-limiting example, the therapeutic nanoparticle is a stealth nanoparticle or a target-specific stealth nanoparticle as described in U.S. Patent Publication No. US20130172406, the contents of which are herein incorporated by reference in their entirety.

In some embodiments, the therapeutic nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In yet another non-limiting example, the lipid nanoparticle comprises the block copolymer PEG-PLGA-PEG (see e.g., the thermosensitive hydrogel (PEG-PLGA-PEG) was used as a TGF-betal gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253, the contents of each of which are herein incorporated by reference in their entirety). The RNA (e.g., mRNA) vaccines of the present disclosure may be formulated in lipid nanoparticles comprising the PEG-PLGA-PEG block copolymer.

In some embodiments, the therapeutic nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In some embodiments, the block copolymers described herein may be included in a polyion complex comprising a non-polymeric micelle and the block copolymer. (see e.g., U.S. Publication No. 20120076836, the contents of which are herein incorporated by reference in their entirety).

In some embodiments, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or U.S. Patent Publication No US20130121954, the contents of each of which are herein incorporated by reference in their entirety. In some embodiments, the poly(vinyl ester) polymers may be conjugated to the polynucleotides described herein.

In some embodiments, the therapeutic nanoparticle may comprise at least one diblock copolymer. The diblock copolymer may be, but it not limited to, a poly(lactic) acid-poly(ethylene)glycol copolymer (see, e.g., International Patent Publication No. WO2013044219, the contents of which are herein incorporated by reference in their entirety).

In certain embodiments of the present disclosure, a subject is administered a single dose of the vaccine or the pharmaceutical formulation as described in the present disclosure. In certain embodiments of the present disclosure, a subject is administered an initial dose of the vaccine or the pharmaceutical formulation followed by one or more booster doses of the vaccine or the pharmaceutical formulation. In certain embodiments of the present disclosure, the vaccine or the pharmaceutical formulation is administered to the subject via intradermal injection or intramuscular injection. In various embodiments, the subject is a human. In various embodiments, the subject is at increased risk of death or serious illness from SARS-CoV-2 infection. In various embodiments, the subject is immunocompromised or has one or more comorbidities that increase the risk of death or serious illness from SARS-CoV-2 infection. In various embodiments, the subject is a pediatric subject. In various embodiments, the administration of the vaccine or the pharmaceutical formulation results in decreased side-effects relative to a comparator vaccine.

In various embodiments, the administration of the vaccine or the pharmaceutical formulation results in decreased negative effects on the nervous system and/or the immune system of the subject relative to a comparator vaccine. In various embodiments, the one or more symptoms are that of long COVID or post-COVID syndromes/conditions (PCC).

In various embodiments, the one or more symptoms are general symptoms selected from tiredness or fatigue that interferes with daily life, symptoms that get worse after physical or mental effort (also known as “post-exertional malaise”), and fever. In various embodiments, the one or more symptoms are SARS-CoV-2 and heart symptoms selected from difficulty breathing or shortness of breath, cough, chest pain, and fast-beating or pounding heart (also known as heart palpitations). In various embodiments, the one or more symptoms are neurological symptoms selected from difficulty thinking or concentrating (sometimes referred to as “brain fog”), headache, sleep problems, dizziness when you stand up (lightheadedness), pins-and-needles feelings, change in smell or taste, and depression or anxiety. In various embodiments, the one or more symptoms are digestive symptoms selected from diarrhea or stomach pain. In various embodiments, the one or more symptoms are selected from Joint or muscle pain, rash, and changes in menstrual cycles.

Exemplary amino sequences of the SARS-CoV-2 virus spike proteins are provided in Table 1.

TABLE 1
SARS-CoV-2 Spike Protein and variants

| SEQ ID | Sequence |
|---|---|
| SEQ ID | MFVFLVLLPLVSSQCVNLTTRTQLPPAYTNSFTRGVYYPDKVFRSSVLHSTQD |
| NO: 1 | LFLPFFSNVTWFHAIHVSGTNGTKRFDNPVLPFNDGVYFASTEKSNIIRGWIFGT |
| Spike | TLDSKTQSLLIVNNATNVVIKVCEFQFCNDPFLGVYYHKNNKSWMESEFRVYSS |
| (UniProt | ANNCTFEYVSQPFLMDLEGKQGNFKNLREFVFKNIDGYFKIYSKHTPINL VRDL |
| Accession | PQGFSALEPLVDLPIGINITRFQTLLALHRSYLTPGDSSSGWTAGAAAYYVGYLQ |
| No. | PRTFLLKYNENGTITDAVDCALDPLSETKCTLKSFTVEKGIYQTSNFRVQPTESIV |
| PODTC2) | RFPNITNLCPFGEVENATRFASVYAWNRKRISNCVADYSVLYNSASFSTFKCYG |
| | VSPTKLNDLCFTNVYADSFVIRGDEVRQIAPGQTGKIADYNYKLPDDFTGCVIA |
| | WNSNNLDSKVGGNYNYLYRLFRKSNLKPFERDISTEIYQAGSTPCNGVEGENCY |
| | FPLQSYGFQPTNGVGYQPYRVVVLSFELLHAPATVCGPKKSTNLVKNKCVNFN |
| | FNGLTGTGVLTESNKKFLPFQQFGRDIADTTDAVRDPQTLEILDITPCSFGGVSVI |
| | TPGTNTSNQVAVLYQDVNCTEVPVAIHADQLTPTWRVYSTGSNVFQTRAGCLI |
| | GAEHVNNSYECDIPIGAGICASYQTQTNSPRRARSVASQSIIAYTMSLGAENSVA |
| | YSNNSIAIPTNFTISVTTEILPVSMTKTSVDCTMYICGDSTECSNLLLQYGSFCTQL |
| | NRALTGIAVEQDKNTQEVFAQVKQIYKTPPIKDFGGFNFSQILPDPSKPSKRSFIE |
| | DLLFNKVTLADAGFIKQYGDCLGDIAARDLICAQKFNGLTVLPPLLTDEMIAQY |
| | TSALLAGTITSGWTFGAGAALQIPFAMQMAYRFNGIGVTQNVLYENQKLIANQ |
| | FNSAIGKIQDSLSSTASALGKLQDVVNQNAQALNTLVKQLSSNFGAISSVLNDIL |
| | SRLDKVEAEVQIDRLITGRLQSLQTYVTQQLIRAAEIRASANLAATKMSECVLG |
| | QSKRVDFCGKGYHLMSFPQSAPHGVVFLHVTYVPAQEKNFTTAPAICHDGKAH |
| | FPREGVFVSNGTHWFVTQRNFYEPQIITTDNTFVSGNCDVVIGIVNNTVYDPLQ |
| | PELDSFKEELDKYFKNHTSPDVDLGDISGINASVVNIQKEIDRLNEVAKNLNESLI |
| | DLQELGKYEQYIKWPWYIWLGFIAGLIAIVMVTIMLCCMTSCCSCLKGCCSCGS |
| | CCKFDEDDSEPVLKGVKLHYT |
| SEQ ID | X₁DSX₂KEEX₃ |
| NO: 2 | X₁ is L, A, V, or G |
| | X₂ is F, A, V, or G |
| | X₃ is L, A, V, or G |
| | In which at least one amino acid represented by X₁, X₂ and X₃ is mutated relative to the |
| | corresponding wild-type amino acid sequence, and X₁, X₂ and X₃ are not L, F, and L, |
| | respectively, in the same polypeptide sequence. |
| SEQ ID | XDSFKEEL |
| NO: 3 | X is A, V, G, or any other natural or synthetic or unnatural or modified amino acid, but |
| | not L. |
| SEQ ID | LDSXKEEL |
| NO: 4 | X is A, V, G, or any other natural or synthetic or unnatural or modified amino acid, but |
| | not F. |
| SEQ ID | LDSFKEEX |
| NO: 5 | X is A, V, G, or any other natural or synthetic or unnatural or modified amino acid, but |
| | not L. |
| SEQ ID | X₁DSX₂KEEL |
| NO: 6 | X₁ is L, A, V, or G, or any other natural or synthetic or unnatural or modified amino |
| | acid. |
| | X₂ is F, A, V, or G, or any other natural or synthetic or unnatural or modified amino |
| | acid. |
| | In which when X₁ is L, X₂ is not F, and when X₂ is F, X₁ is not L. |
| SEQ ID | X₁DSFKEEX₂ |
| NO: 7 | X₁ is L, A, V, or G, or any other natural or synthetic or unnatural or modified amino |
| | acid. |
| | X₂ is L, A, V, or G, or any other natural or synthetic or unnatural or modified amino |
| | acid. |
| | In which when X₁ is L, X₂ is not L, and when X₂ is L, X₁ is not L. |
| SEQ ID | LDSX₁KEEX₂ |
| NO: 8 | X₁ is F, A, V, or G, or any other natural or synthetic or unnatural or modified amino |
| | acid. |
| | X₂ is L, A, V, or G, or any other natural or synthetic or unnatural or modified amino |
| | acid. |
| | In which when X₁ is F, X₂ is not L, and when X₂ is L, X₁ is not F. |
| SEQ ID | ADSAKEEA |
| NO: 9 | |
| SEQ ID | LAKILEEV |
| NO: 10 | |
| α7 | |
| SEQ ID | X₁ X₂ X₃ X₄ X₅ X₆ X₇ X₈ |
| NO: 19 | In which when X₁ is L, X₄ is not For, independently, X₈ is not L; when X₄ is F, X₁ is |
| | not L or, independently, X₈ is not L; when X₈ is L, X₁ is not L or, independently, X₄ is |
| | not F; |
| | further in which when, independently, X₂ is D, X₁ is not L, X₃ is not S, X₄ is not F, X₅ |
| | is not K, X₆ is not E, X₇ is not E, or X₈ is not L; |
| | when X₃ is S, independently, X₁ is not L, X₂ is not D, X₄ is not F, X₅ is not K, X₆ is not |
| | E, X₇ is not E, or X₈ is not L; |
| | when X₅ is K, independently, X₁ is not L, X₂ is not D, X₃ is not S, X₄ is not F, X₆ is not |
| | E, X₇ is not E, or X₈ is not L; |
| | when X₆ is E, independently, X₁ is not L, X₂ is not D, X₃ is not S, X₄ is not F, X₅ is not |
| | K, X₇ is not E, or X₈ is not L; |
| | when X₇ is E, X₁ is not L, X₂ is not D, X₃ is not S, X₄ is not F, X₅ is not K, X₆ is |
| | not E, or X₈ is not L; |
| | with the proviso that each of X₁, X₂, X₃, X₄, X₅, X₆, X₇, and X₈ independently, is any |
| | other natural or synthetic or unnatural or modified amino acid. |
| SEQ ID | LXSFKEEL |
| NO: 20 | In which X is not D, but any other natural or synthetic or unnatural or modified amino |
| | acid |
| SEQ ID | LDXFKEEL |
| NO: 21 | In which X is not S, but any other natural or synthetic or unnatural or modified amino |
| | acid |
| SEQ ID | LDSFKXEL |
| NO: 22 | In which X is not E, but any other natural or synthetic or unnatural or modified amino |
| | acid |
| SEQ ID | LDSFKEXL |
| NO: 23 | In which X is not E, but any other natural or synthetic or unnatural or modified amino |
| | acid |

### Vaccines and Pharmaceutical Compositions

Vaccines, as provided herein, comprise at least one (one or more) ribonucleic acid (RNA) (e.g., mRNA) polynucleotide having an open reading frame encoding at least one antigenic polypeptide SARS-CoV-2 antigenic polypeptides. The term “nucleic acid” includes any compound and/or substance that comprises a polymer of nucleotides (nucleotide monomer). These polymers are referred to as polynucleotides. Thus, the terms “nucleic acid” and “polynucleotide” are used interchangeably.

Nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

A vaccine as described herein may further comprise an adjuvant, preservative, stabilizer and/or a carrier. Exemplary preservatives, carriers, and stabilizers are described below. Exemplary adjuvants include, without limitation, aluminum compounds (e.g., aluminum hydroxide, aluminum phosphate, aluminum hydroxyphosphate, oxyhydroxide, orthophosphate, sulphate) or mixtures thereof, MF59 (5% Squalene, 0.5% Tween 80, and 0.5% Span 85), liposomes, ISCOMs, SAF, containing 10% Squalane, 0.4% Tween 80, 5% pluronic-block polymer L121, and thr-MDP, Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), saponin adjuvants, such as QuilA or QS21, chitosan, complete Freund's adjuvant (CFA) and incomplete Freund's adjuvant (IFA), cytokines, such as interleukins (e.g., IL-1, IL-2, IL-4, IL-5, IL-6, IL-7, IL-12), interferons (e.g., interferon-γ), macrophage colony stimulating factor, tumor necrosis factor, monophosphoryl lipid A (MPL) or 3-O-deacylated MPL (3dMPL), combinations of 3dMPL with, for example, QS21 and/or oil-in-water emulsions, oligonucleotides comprising CpG motifs, a polyoxyethylene ether or a polyoxyethylene ester, a polyoxyethylene sorbitan ester surfactant in combination with an octoxynol or a polyoxyethylene alkyl ether or ester surfactant, in combination with at least one additional non-ionic surfactant, an immuno-stimulatory oligonucleotide and a saponin, an immuno-stimulant and a metal salt, a saponin and an oil-in-water emulsion, a saponin and 3dMPL and IL12, optionally with a sterol, E. coli heat-labile enterotoxin (LT), or detoxified mutants thereof, such as the K63 or R72 mutants; (U) cholera toxin (CT), or diphtheria toxin (DT) or detoxified mutants thereof, double-stranded RNA, monophosphoryl lipid A mimics, e.g., aminoalkyl glucosaminide phosphate derivatives, polyphosphazene (PCPP), or a bioadhesive, e.g., esterified hyaluronic acid microspheres or a mucoadhesive such as crosslinked derivatives of poly(acrylic acid), polyvinyl alcohol, polyvinyl pyrollidone, polysaccharides, and carboxymethylcellulose.

The pharmaceutical compositions and vaccines described herein can be prepared by admixing a quantity of phage with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions and vaccines containing recombinant nucleic acid, protein, or polypeptide disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form will depend upon the intended mode of administration and therapeutic application.

In some embodiments, the vaccine or pharmaceutical composition described herein is administered via oral, subcutaneous injection, intradermal injection, inhalation spray, intraperitoneal injection, intramuscular injection, or intravenous injection. Other methods of administration of a vaccine or pharmaceutical composition are known to those of skill in the art. In some embodiments, the vaccine or pharmaceutical composition is administered to the subject via intravenous administration. In some embodiments, a vaccine as described herein may be administered as a vaccine against a single virus, e.g., a coronavirus, e.g., SARS-CoV-2. In some embodiments, a vaccine as described herein may be administered in a combination vaccine, e.g., in a vaccine against multiple viral infections. In some embodiments, a vaccine as described herein may be administered in combination with another vaccine for SARS-CoV-2, e.g., an mRNA-based vaccine (e.g., the Pfizer-BioNTech vaccine “Comirnaty”, the Moderna-NIAID vaccine), an adenovirus-based vaccine (e.g., the Johnson and Johnson vaccine, the Oxford-AstraZeneca vaccine), or a protein-adjuvant vaccine (e.g., the Novavax vaccine). In certain embodiments, a vaccine as described herein is administered subsequent to a dose of an mRNA-based vaccine (e.g., the Pfizer-BioNTech vaccine “Comirnaty”, the Moderna-NIAID vaccine), an adenovirus-based vaccine (e.g., the Johnson and Johnson vaccine, the Oxford-Astra7eneca vaccine), or a protein-adjuvant vaccine (e.g., the Novavax vaccine). In certain embodiments, a vaccine as described herein is administered subsequent to two doses of an mRNA-based vaccine (e.g., the Pfizer-BioNTech vaccine “Comirnaty”, the Moderna-NIAID vaccine), an adenovirus-based vaccine (e.g., the Johnson and Johnson vaccine, the Oxford-AstraZeneca vaccine), or a protein-adjuvant vaccine (e.g., the Novavax vaccine). In certain embodiments, a vaccine as described herein is administered subsequent to three doses of an mRNA-based vaccine (e.g., the Pfizer-BioNTech vaccine “Comirnaty”, the Moderna-NIAID vaccine), an adenovirus-based vaccine (e.g., the Johnson and Johnson vaccine, the Oxford-AstraZeneca vaccine), or a protein-adjuvant vaccine (e.g., the Novavax vaccine). In certain embodiments, a vaccine as described herein is administered prior to administration of an mRNA-based vaccine (e.g., the Pfizer-BioNTech vaccine “Comirnaty”, the Moderna-NIAID vaccine), an adenovirus-based vaccine (e.g., the Johnson and Johnson vaccine, the Oxford-AstraZeneca vaccine), or a protein-adjuvant vaccine (e.g., the Novavax vaccine).

A vaccination schedule may be used to administer said vaccine to a subject. An exemplary vaccination schedule may be a dose of an initial, priming vaccine, followed by one or more doses of a booster vaccine. As used herein, a “booster vaccine” is an additional administration of a vaccine after an initial dose, offering re-exposure to the immunizing antigen after immunity to the antigen may have decreased. In some embodiments, the initial vaccine and the booster vaccine are at the same dose. In some embodiments, the booster vaccine is at lower dose than the initial vaccine. In some embodiments, a booster vaccine may be administered, for example, about every 1 year, about every 2 years, about every 3 years, about every 4 years, about every 5 years, about every 6 years, about every 7 years, about every 8 years, about every 9 years, about every 10 years, or about more than 10-years between booster vaccines. In some embodiments, a vaccine may be administered as a single dose.

### Spike Protein Ectodomain S12 and α7nAChR Expression

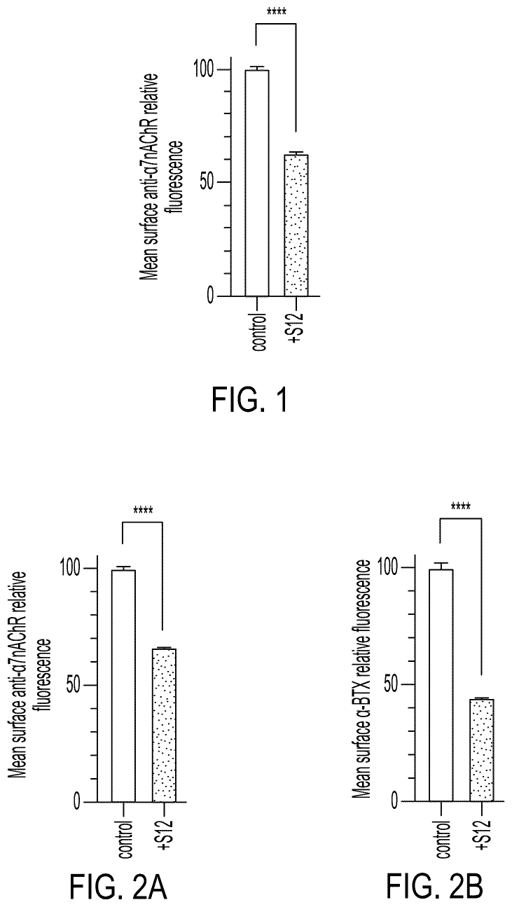

One of the discoveries from the current disclosure was the downregulation of surface α7nAChR by the spike protein ectodomain S12. The extent of the downregulation is profound, with more than one third of surface α7nAChR and more than half of the functional α7nAChR (FIGS. 1 and 2). The α7nAChR downregulation by S12 may be ubiquitous across different cell types because it has happened in both neuronal PC12 cells and HEK293 cells expressing native and recombinant α7nAChR (FIGS. 1 and 2), respectively. It is known that α7nAChR is expressed in various cells and has functions in both neuronal and immune systems (Bencherif et al., 2011; Corradi and Bouzat, 2016; Letsinger et al., 2022; Pavlov and Tracey, 2017; Schloss et al., 2022; Wang et al., 2003). A significant depletion of functional α7nAChR, such as that caused by co-expression of S12 (FIG. 2), would have a negative impact on neuronal circuitry and regulation of anti-inflammatory signaling (Cheng and Yakel, 2015; Corradi and Bouzat, 2016; Freedman et al., 1995; Gotti et al., 2006a; Koukouli and Maskos, 2015; Lange et al., 1993; Mizrachi et al., 2021). Deficiency of functional α7nAChR in the hippocampus and other brain regions is associated with neuropsychiatric disorders with numerous deleterious symptoms, including cognitive impairments, and sensory processing deficits (Freedman et al., 1995; Gotti et al., 2006a; Lange et al., 1993). Enhancing α7nAChR function, on the other hand, can improve cognitive performance, attention, and memory (Corradi and Bouzat, 2016). α7nAChR is a key player in the cholinergic anti-inflammatory pathway (Wang et al., 2003). Its presence in immune cells is critical for α7nAChR-mediated anti-inflammatory signaling and neuroprotection (Egea et al., 2015). Thus, a significant reduction of α7nAChR can potentially impair not only cognitive performance and sensory processing (Freedman et al., 1995; Gotti et al., 2006a; Lange et al., 1993), but also normal immune response to inflammation (Egea et al., 2015). The discovered S12 effect on the downregulation of surface α7nAChR provides a new perspective for understanding certain symptoms of COVID-19, particularly cognitive and immunity-related symptoms of long COVID (Boix and Merino, 2022; Huang et al., 2021; Nalbandian et al., 2021; Phillips and Williams, 2021; Premraj et al., 2022).

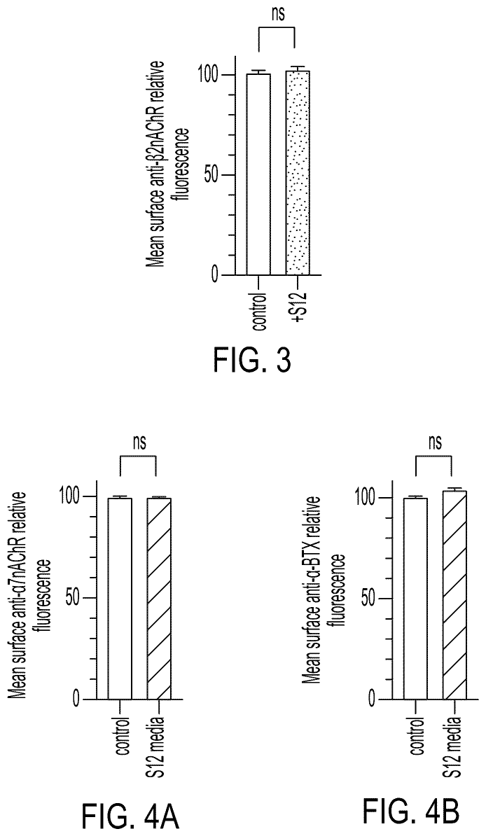

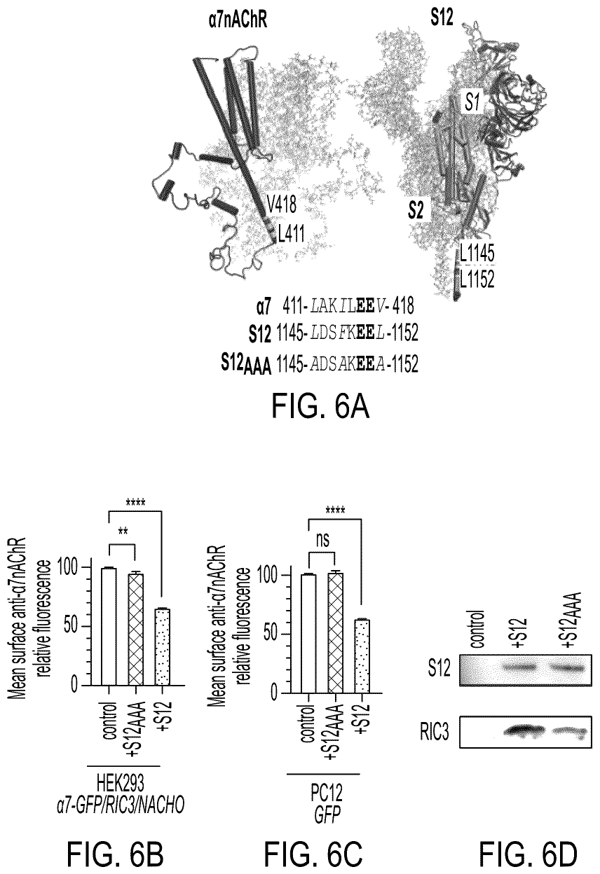

Another major discovery from the current disclosure is the underlying mechanism of how S12 downregulates surface α7nAChR. It is intriguing to learn that S12 contains a helical segment (L1145-L1152) homologous to the α7nAChR helical segment (L411-V418) responsible for binding to chaperones for receptor assembly and trafficking. It is even more intriguing to see that eliminating the possibility for S12 binding the α7nAChR chaperones (RIC-3 and anti-apoptotic Bcl-2 proteins) in S12_{AAA} restored surface α7nAChR close to normal (FIGS. 6B, 6C). The uncovered competition mechanism for downregulating surface α7nAChR by S12 also explains why S12 co-expression did not affect the surface population of α4β2nAChRs (FIG. 3), which are largely insensitive to regulation by anti-apoptotic Bcl-2 proteins due to the lack of bulky hydrophobic residues critical for binding the chaperones found in α7nAChR (Dawe et al., 2019). Interestingly, competition for host factors may be a general mechanism for S12 pathogenesis. A recent study found inhibition of epithelial sodium channels (ENaC) when mRNAs of ENaC and S12 were co-injected and expressed in Xenopus oocytes and the inhibition was most likely due to S12 competition for host factors (Grant and Lester, 2021).

The segment of L1145-L1152 is conserved in all variants of SARS-CoV-2, including the newest omicron variant. A negative impact resulting from this S12 segment binding to the chaperone proteins, especially anti-apoptotic Bcl-2 proteins, may go beyond downregulation of surface α7nAChR. The main function of anti-apoptotic Bcl-2 proteins is to directly bind pro-apoptotic BH3-only proteins and consequently to restrain pro-apoptotic BAX/BAK co-assembly, which is a requisite gateway to mitochondrial dysfunction and death (Wei et al., 2001; Youle and Strasser, 2008). Sequence homology exists between the L1145-L1152 segment of S12 and the BH3-motif responsible for binding anti-apoptotic Bcl-2 proteins. It is conceivable that competition of S12 with the BH3-motif for binding anti-apoptotic Bcl-2 proteins will directly weaken actions of Bcl-2 proteins in restraining pro-apoptotic BAX/BAK co-assembly, imposing detrimental effects on mitochondrial function and immune cell survival (Wei et al., 2001). Altogether, the potential negative impact of S12 through its L1145-L1152 segment on cell apoptosis and suppression of surface α7nAChR warrants further investigations. The current SARS-CoV-2 mRNA vaccines encode the full-length spike protein (Corbett et al., 2020; Walsh et al., 2020), including the L1145-L1152 segment. The encoded proteins have the same sequence as the one in the SARS-CoV-2 genome (NC_045512.2) with the exception of two mutations (K986P, V987P) for improving the stability and efficacy of the vaccines (Corbett et al., 2020; Walsh et al., 2020). It should be considered whether to mutate the L1145-L1152 segment, as we did for S12_{AAA}, in new versions of SARS-CoV-2 mRNA-based vaccines to prevent potential adverse effects caused by this segment.

A potential contribution of α7nAChR to COVID-19 pathophysiology was proposed based on the sequence homology found in S12 and snake venom neurotoxins, including α-BTX (Changeux et al., 2020), which are potent competitive antagonists of α7nAChR. The predicted interactions were demonstrated by computational studies between α7nAChR and a S12 peptide (Y674-R685) homologous to α-BTX (Oliveira et al., 2021). A recent report showed experimental evidence supporting the S12 peptide interactions with α7nAChR and functional impact of the interactions on α7nAChR (Chrestia et al., 2022). Isolated S1 (at a concentration as low as 1 nM) was recently found to inhibit currents of Xenopus oocytes expressing α7nAChR (Farley and Anderson, 2022). Thus far, however, no experimental result shows direct interactions between the intact S12 trimer and α7nAChR. In the current study, we did not observe a significant change in the α-BTX staining of α7nAChR when S12 was present in cell culture media (Supplementary FIG. 1). Steric hindrance of its bulky trimer may have prevented S12 from access to a potential binding site of α7nAChR or S12 simply could not compete with high-affinity α-BTX binding. Nevertheless, a more comprehensive functional study is required to define a role of an intact S12 trimer in the channel function of α7nAChR.

How long can S12 remain in the human body to potentially contribute to post-infection symptoms? The elevated level of immunoreactive S12 was detected in the serum of COVID-19 patients aged 46 or older between 5 and 10 days from the onset of COVID-19 symptoms (Avolio et al., 2021). The prolonged S12 exposure prompted pericyte dysfunction and contributed to microvascular injury through a potentially non-infective mechanism of COVID-19 microvascular disease (Avolio et al., 2021). According to the United States Center for Disease Control and Prevention, patients who have recovered from COVID-19 can have detectable SARS-CoV-2 RNA in upper SARS-CoV-2 specimens for up to 3 months after onset of illness. Detection of sub-genomic SARS-CoV-2 RNA has been reported in moderately or severely immunocompromised patients up to >140 days after a positive SARS-CoV-2 test result (Choi et al., 2020; Weigang et al., 2021). The circumstances resulting in persistently detectable SARS-CoV-2 RNA and how the detectable RNA contributes to long-COVID have yet to be determined. However, the longevity of SARS-CoV-2 RNA raises a significant concern for patients recovered from COVID-19 and patients chronically immunocompromised.

### EXAMPLES

The following examples are merely illustrative and are not intended to limit the scope or content of the invention in any way.

### Example 1

### Cell Culture

PC12 cells (male, rat adrenal gland pheochromocytoma, RRID:CVCL_0481) were obtained from the ATCC (ATCC Cat#CRL-1721) and maintained at 37° C. and 5% CO₂ in a humidified incubator. Growth medium was F12K (Thermo Fisher Cat# 21127-030) supplemented with 10% Horse Serum (Thermo Fisher Cat# 26050088), 5% Fetal Bovine Serum (Thermo Fisher Cat# 16140071), and 1% penicillin/streptomycin (Cytiva Cat# SV30010) on collagen-coated plates or glass coverslips. Cells were maintained at low passage number from the source but were not otherwise authenticated during these experiments. For differentiation, PC-12 cells were treated for 4 days in F12K media containing 1% Horse Serum and 100 ng/ml 2.5S nerve growth factor (Thermo Fisher Cat# 13257-019) and 1% penicillin/streptomycin. PC12 cells were transfected using the Transporter 5 Transfection Reagent (PolySciences Cat# 26008-5) following the manufacturer's instructions. Transfected cells comprised three experimental groups: control (transfected with mVenus C1 (RRID:Addgene_27794)), +S12 (co-transfected with mVenus C1 and pαH-S-RRAR (“RRAR” disclosed as SEQ ID NO: 24) (RRID:Addgene_164569)) and +S12_{AAA} (co-transfected with mVenus C1 and pαH-S-AAA, 1:1). Cells were maintained at low passage number from the source but were not otherwise authenticated during these experiments.

HEK293T/17 cells (female, human fetal kidney, RRID:CVCL_1926) were obtained from the ATCC (ATCC Cat#CRL-11268) and maintained at 37° C. and 5% CO₂ in a humidified incubator. Growth medium was DMEM, high glucose, pyruvate (Gibco Cat# 11995-065) supplemented with 10% Fetal Bovine Serum (Thermo Fisher Cat#16140071), and 1% penicillin/streptomycin (Cytiva Cat#SV30010). Cells were maintained at low passage number from the source but were not otherwise authenticated during these experiments. For imaging studies, cells were grown on collagen-coated 24 well plates or glass coverslips. HEK293T/17 cells were transfected using the DOTAP Liposomal Transfection Reagent (Roche Cat# 11202375001) following the manufacturer's instructions. Transfected cells comprised three experimental groups: control (co- transfected with pLenti6-α7nAChR-ZsG, pLX304-RIC3 (DNASU Cat#HsCD00438164), and pCMV3-TMEM35 (NACHO), 3:2:1), +S12 (co-transfected with pLenti6-α7nAChR-ZsG, pLX304-RIC3, pCMV3-TMEM35 (NACHO, Sino Biological Cat# HG27483-UT), and pαH-S-RRAR (“RRAR” disclosed as SEQ ID NO: 24), 3:2:1:3) and +S12AAA (co-transfected with pLenti6-α7nAChR-ZsG, pLX304-RIC3, pCMV3-TMEM35 (NACHO), and pαH-S-AAA (RRID:Addgene_164569), 3:2:1:3).

Spike HexaPro protein was expressed in Expi293 GnTI- cells (female, human fetal kidney, RRID:CVCL_B0J7) grown in baffled flasks with Expi293 Expression Medium (Gibco Cat# A14351-01) at 37° C., 120 rpm and 8% CO2 in a humidified atmosphere. Expi293 cells were transfected as follows: cells were diluted to 2.5×10⁶ cell/ml and grown 20-24 h. SARS-CoV-2 S HexaPro (RRID:Addgene_154754, 1.5 μg/mL final culture volume) was diluted in 1/20th final culture volume with media. Linear polyethylenimine MW 2500 (Polysciences Cat# 23966-1, 4.5 μg/mL final culture volume) was diluted in 1/20th final culture volume with media. The DNA and polyethylenimine solutions were combined and incubated at room temperature 30 min before being added to the overnight culture with a final cell density of 2.5×10⁶ cell/ml. The culture was then grown overnight before stimulating protein production with the addition of 2.2 mM valproic acid (Thermo Fisher Cat# A12962). Cells were grown for an additional 4 days and the conditioned media containing Spike HexaPro protein was harvested.

KXα4β2 stably transfected HEK293 cells[15016836] were maintained and transfected as described above for HEK293T/17, except 0.7 mg/ml G418 (Sigma Cat# G8168) was added to maintain selection of α4β2 nAChR. Cells were maintained at low passage number from the source but were not otherwise authenticated during these experiments. Transfected cells comprised two experimental groups: control (transfected with pcDNA3-YFP (RRID:Addgene_13033)) and +S12 (co-transfected with pcDNA3-YFP and pαH-S-RRAR (“RRAR” disclosed as SEQ ID NO: 24) (RRID:Addgene_164569), 1:1).

### Plasmids

Human α7nAChR was subcloned from pMXT-α7AChR (a gift from J. Lindstrom[8145738]) with primers α7_fwd (tctagaggatcgtcgccaccatgcgctgctcgccggga) (SEQ ID NO:11) and α7_rev (ctagactcgatgatcagttactacgcaaagtctttggacacggcc) (SEQ ID NO:12) to the mammalian dual expression vector pLenti6-CMV-RFPn-CMV-ZsG (a gift from Bing Wang, University of Pittsburgh) replacing RFPn using overlapping PCR[20569222] with primers v_fwd (taactgatcatcgagtctagaggg) (SEQ ID NO:13) and v_rev (ggtggcgacgatcctctaga) (SEQ ID NO:14) to create pLenti6- -α7nAChR-ZsG. pLX304-RIC3 was obtained from DNASU (clone HsCD00438164). pCMV3-TMEM35 (NACHO) was purchased from Sino Biological (cat# HG27483-UT). SARS-CoV-2 S HexaPro (stabilized S12, RRID:Addgene_154754[32703906]), pαH-S-RRAR (“RRAR” disclosed as SEQ ID NO: 24) (wild-type S12, RRID:Addgene_164569[33417835]), mVenus C1(GFP variant, RRID:Addgene_27794[17040988]), and pcDNA3-YFP(RRID:Addgene_13033, Doug Golenbock) were obtained from Addgene. pαH-S-AAA was constructed from pαH-S-RRAR (“RRAR” disclosed as SEQ ID NO: 24) by PCR mutagenesis using the following primers S12AAA begin_fwd (tgtgctgaacgatatcctgtctagactggacaaggtgg) (SEQ ID NO:15) S12AAA begin rev (ggcctcttccttggcagagtcggcctcaggttgcagagggtc) (SEQ ID NO:16) S12AAA end_fwd (gccgactctgccaaggaagaggccgacaagtactttaaaaaccacaccagcc) (SEQ ID NO:17) S12AAA end_rev (ctatgaccatgattacgccaagcttgggctgcaggtcg) (SEQ ID NO:18).

### Immunocytochemistry and Fluorescent Labeling

Transfected cells were expressed for ˜36 hours and washed with media or Dulbecco's phosphate buffered saline (DPBS) before being labeled. For bungarotoxin live labeling, α-bungarotoxin Alexa Fluor 594 conjugate (Invitrogen Cat# B13423) was added at 5 μg/ml media and incubated at 37° C. for 30 min, then washed three times with DPBS and fixed for imaging with 4% paraformaldehyde (PFA). For immunocytochemistry, the washed cells were fixed in 4% PFA and then treated with R&D Acidic Antigen Retrieval Reagent (R&D Cat# CTS014) for 5 minutes at 90° C. The cells were incubated with blocking buffer (10 mg/ml bovine serum albumin and 5% goat serum (Sigma Cat# G9023) in DPBS) for 1 h and incubated with primary antibody in blocking buffer at 4° C. overnight. After washing three times in DPBS the cells were incubated with 1:850 Alexa Fluor-594 conjugated secondary anti-rabbit antibody (Thermo Fisher Cat# A-11012; RRID:AB_2534079) in blocking buffer for 2 h at room temperature. After washing 3× in DPBS, the cells were fixed and ready for imaging. Primary antibodies were anti-α7nAChR Polyclonal (RRID:AB_2900286), SARS-CoV-2 Spike Protein (RBD) Polyclonal (RRID:AB_2890581) or CHRNB2 Polyclonal (RRID:AB_2735656) as indicated in the figure legends. Where indicated, cells were permeabilized by including 0.05% Tween 20 (Fisher Cat# BP337-100) in the antigen retrieval reagent and 0.2% Triton X-100 (Sigma Cat# T8787-100ML) in the blocking buffer and antibody incubations. All fluorescence images were collected using uniform exposure settings with an Olympus IX-81 microscope system and SlideBook 6.0.22 Digital Microscopy Software (3i) for data collection and digitization. GFP or YFP fluorescence was used to identify transfected cells and the intensity of Alexa Fluor-594 staining for each transfected cell was measured. After background subtraction the intensity of each cell was normalized to the mean intensity of the control group. Each experiment was repeated the number of times indicated in the figure legends.

### Protein Purification

After 5 days expression the conditioned media of cell cultures transfected with SARS-CoV-2 S HexaPro (stabilized S12, RRID:Addgene_154754[32703906]) were harvested and filtered before loading onto a 5 mL StrepTrap XT column (Cytiva Cat# 29401322), which was then washed with 50 mM Tris pH 8 and 150 mM NaCl. S12 was eluted with 50 mM biotin (Alfa Aesar Cat#A14207.09), yielding ˜25 mg of high-purity S12 per liter of culture media. Trimeric S12 was isolated using size exclusion chromatography with a Superose 6 Increase 10/300 GL column (Cytiva Cat# 29-0915-96) equilibrated with 20 mM Tris pH 8 and 150 mM NaCl, resulting in ˜12 mg trimeric S12 per liter of culture media.

### Protein Electrophoresis and Western Blot

Conditioned media from cells co-transfected with pαH-S-RRAR (“RRAR” disclosed as SEQ ID NO: 24) (wild-type S12, RRID:Addgene_164569 [33417835]) and mVenus C1(GFP variant, RRID:Addgene_27794 [17040988]) or transfected with mVenus C1 alone were collected after 5 days expression. 15 μL conditioned media from each group was subjected to electrophoresis on a 10% Laemmli SDS-PAGE gel. Proteins were then transferred to a 0.2 μm PVDF membrane at 100 mA overnight in Tris-glycine pH 9.2, 20% methanol at 4° C. The membrane was then incubated in blocking buffer (5% bovine serum albumin in Tris buffered saline supplemented with 0.1% Tween-20 (TBST)) for 1 hour at room temperature with gentle rocking. The membrane was then incubated with the primary antibody (SARS-CoV-2 Spike Protein (RBD) Polyclonal Antibody (Thermo Fisher, Cat# PAS-114451; RRID:AB_2890581) diluted 1:250 in blocking buffer) overnight at 4° C. with gentle rocking. After washing 4× in TBST for 5 min each, the membrane was incubated with the secondary antibody (Goat Anti-rabbit IgG HRP linked (Cell Signaling Technology Cat# 7074; RRID:AB_2099233), diluted 1:2000 in blocking buffer) overnight at 4° C. with gentle rocking. After washing 4× in TBST for 5 min each, the membrane was developed with SuperSignal West Pico Plus Chemiluminescent Substrate (Thermo Fisher Cat# 34577) according to the manufacturer's instructions.

### Cryo-EM

Cryo-EM was performed using a Titan Krios cryo-electron microscope equipped with a Falcon 3 direct electron detector in the cryo-EM facility at the University of Pittsburgh School of Medicine. Briefly, Quantifoil 1.2/1.3 Au 300 mesh grids were glow discharged for 30 s at 25 mA. 3 μl of S12 (0.5 mg/mL) was applied to the grids and blotted for 3-4 s followed by plunge freezing into liquid ethane using a Vitrobot Mark IV. Data were collected on the Titan Krios using EPU at 0.832 Ans/pixel. Approximately 100 micrographs were collected, drift corrected, and autopicked using the blob picker in CryoSPARC 6. Particles were sorted using two rounds of 2D classification followed by homogeneous refinement with C3 symmetry in CryoSPARC using a low-pass filtered volume of EMD-11334 as the initial reference.

### Quantification and Statistical Analysis

The total number of cells analyzed from each experimental group and the number of times each experiment was repeated is indicated in the figure legends. Significance was determined by two-tailed unpaired t test or one-way ANOVA with Dunnett's multiple comparisons using Prism 9.4.0 software (GraphPad). A value of p<0.05 was considered significant.

### Expression of S12 in PC12 Cells Suppresses Native Surface α7nAChR

PC12 cells contain native α7nAChR and downstream signaling proteins, and are well-established and commonly used for studies of neuroinflammation (Sangaran et al., 2020). Surface expression of α7nAChR was measured in intact non-permeabilized PC12 cells using immunocytochemistry with anti-α7nAChR primary antibody and an Alexa Fluor 594 conjugated secondary antibody. Transfected cells were identified by the expression of a green fluorescent protein (mVenus), either alone (control) or in the presence of S12 (+S12). The mean intensity of the α7nAChR staining for each transfected cell was quantified and normalized by the mean intensity for the control group. The PC12 cells co-transfected with plasmids expressing mVenus+S12 showed ˜37% reduction in cell surface α7nAChR relative to those in the control group (FIG. 1).

### Co-Expression of S12 with α7nAChR Suppresses Surface Expression of α7nAChR in HEK293 Cells

HEK293 cells show negligible α7nAChR expression unless in the presence of protein chaperones to promote α7nAChR assembly and biogenesis (Dawe et al., 2019; Gu et al., 2016; Halevi et al., 2003; Matta et al., 2021). This feature enables investigations into S12 effects on recombinant α7nAChR expression. HEK293 cells were transfected with plasmid constructs expressing α7nAChR with or without co-expression of S12 in addition to cDNAs encoding the green fluorescent protein ZsGreen and the chaperones. The transfected cells showed robust surface expression of α7nAChR as measured by labeling with anti-α7nAChR antibody or with an Alexa Fluor 594 conjugate of α-bungarotoxin (αBTX), which is an α7nAChR-selective antagonist and its binding indicates well-folded functional α7nAChR (Couturier et al., 1990). Compared to the control group, co-expression of S12 in HEK293 cells reduced the total surface α7nAChRs by ˜35% (FIG. 2A) and functional α7nAChR by ˜57% (FIG. 2B).

### S12-Caused Suppression of Surface nAChR is Receptor Subtype Specific

Does S12 expression also downregulate other nAChRs in addition to α7nAChRs? To answer this question, surface expression of α4β2nAChR, a major subtype of nAChRs in the brain (Gotti et al., 2006b), was examined. In contrast to what was observed on α7nAChRs, S12 co-expression had no impact on cell surface expression of α4β2nAChR (FIG. 3). The distinctly different responses from α7nAChR and α4β2nAChR suggested that S12's influence on surface receptor expression is receptor subtype dependent.

### Suppression of Functional α7nAChR on Cell Surface Does Not Result from S12 in Extracellular Media

The spike protein ectodomain S12 is largely soluble and can be purified from cell culture supernatant (Gobeil et al., 2021). Could S12 suppress surface α7nAChR through an extracellular action? Interestingly, expression of surface α7nAChR and its binding to αBTX were unaffected by external S12, either through adding ˜400 nM of the purified HexaPro trimer (a stabilized S12 variant) to the culture media or through replacing the culture media of α7nAChR with the conditioned media from cells expressing S12. To completely rule out the possibility of an extracellular S12 action, we utilized the Transwell (Corning) cell culture condition to physically separate cells expressing α7nAChR from those expressing S12 but allow the secreted S12 to pass the porous membrane (0.4 μm) of the Transwell to bathe cells expressing α7nAChR. Again, expression of surface α7nAChR (FIG. 4A) and αBTX binding (FIG. 4B) were unaffected even after being cultured in Transwell for up to 5 days, suggesting that S12 in extracellular media does not play a role in downregulating surface α7nAChR and disrupting aBTX binding to α7nAChR.

### S12 Expression Has Almost No Effect on Intracellular α7nAChR Stores

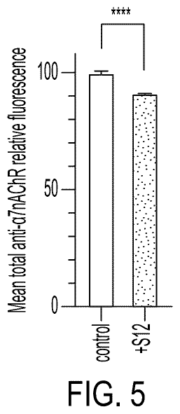

Downregulation of surface α7nAChR could result from a change in receptor trafficking or a decrease of intracellular α7nAChR stores. To differentiate these possibilities, immunocytochemistry with anti-α7nAChR antibody was performed after cell permeabilization, and the total reduction of α7nAChR expression due to S12 co-expression in HEK293 cells was measured. Under these conditions, anti-α7nAChR labeling shows only ˜9% reduction of total α7nAChRs (FIG. 5), less than ⅓ of the ˜35% reduction of surface α7nAChRs (FIG. 2A). Given the known distributions of surface (34±3%) and intracellular (66±3%) receptors (Riganti et al., 2005), the results suggest that co-expression of S12 has no impact on the intracellular α7nAChR pool.

### A S2 Helical Segment Homologous to the Chaperone-Binding Motif of α7nAChR is Required to Downregulate Surface α7nAChR

Chaperone proteins of α7nAChR, including neuronal-specific transmembrane protein 35a (NACHO) (Gu et al., 2016), resistance to inhibitor of cholinesterase-3 (RIC3) (Millar, 2008), and anti-apoptotic B-cell lymphoma 2 (Bcl-2) proteins (Dawe et al., 2019), play a critical role in assembly, trafficking, and ultimately surface expression of α7nAChR. NACHO exerts its action on α7nAChR expression without directly interacting with α7nAChR (Kweon et al., 2020), but Bcl-2 and RIC3 interact directly with an intracellular helical segment (L411-V418) of α7nAChR (FIG. 6A). The α7nAChR mutations by replacing large-size hydrophobic residues with alanine in this segment (L411A-I414A-V418A) reduced surface expression of the receptor due to poor interactions of the mutant with anti-apoptotic Bcl proteins (Dawe et al., 2019). The same helical segment was also found responsible for α7nAChR expression promoted by the chaperone protein RIC3, as mutations of L411A or V418A abolished or largely weakened RIC3-mediated expression of α7nAChR (Castillo et al., 2005). It is intriguing that S12 contains a homologous helical segment (L1145-L1152) (FIG. 6A), which can compete with α7nAChR for binding to the chaperone proteins and lead to suppression of surface α7nAChR. To test this competing hypothesis, this S12 segment was mutated (named S12_{AAA}) by replacing the large-size hydrophobic residues with alanine (L1145A-F1148A-L1152A) (FIG. 6A). S12_{AAA} co-expression imposed only ˜5% reduction of surface α7nAChR, much smaller than that caused by S12 co-expression (FIG. 6B), even though S12_{AAA} expression level is ˜9% higher than S12 expression in HEK cells. In PC12 cells, expression of S12_{AAA} did not downregulate native α7nAChR expression on the cell surface (FIG. 6C). These results led to the conclusion that the helical segment of L1145-L1152 is essential for downregulating surface α7nAChR. The sequence homology between this S12 segment and the chaperone-binding motif of α7nAChR may have enabled S12 to compete with α7nAChR for binding chaperones, and consequently led to a decrease of surface α7nAChR. Pulldown experiments were performed on HEK293 cells coexpressing RIC3 and S12 or S12_{AAA} to test whether S12 competes for binding chaperone proteins and thereby weakens their effects on α7nAChR surface expression. RIC3 pulldown by S12 supported S12 binding to RIC3 and confirmed that the helical segment (L1145-L1152) of S12 is involved in the binding (FIG. 6D). Relative to S12, S12_{AAA} showed reduced RIC3 binding under the same experimental condition in the same assay, with an approximately 67% decrease in RIC3 binding to S12_{AAA} vs S12 observed based on the integrated intensity using ImageJ. Taken together, these results suggest that the L1145-L1152 segment in the spike neck downregulates surface α7nAChR expression. The sequence homology between this S12 segment and the chaperone-binding motif of α7nAChR enables S12 to compete with α7nAChR for binding chaperones and consequently leads to a decreased surface expression of α7nAChR.

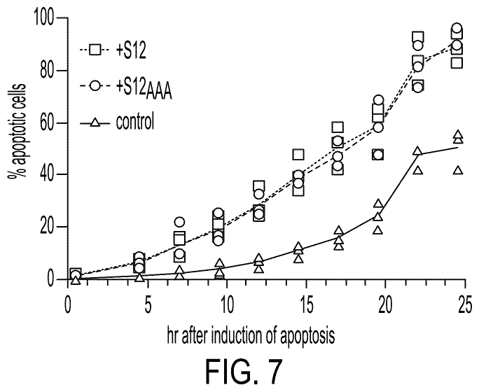

To test whether S12 expression could accelerate cell apoptosis caspase-3/7-dependent apoptosis was measured in differentiated PC12 cells expressing S12 or S12_{AAA} relative to the control. The rate of apoptosis was monitored over 24 h by measuring caspase-3/7 activity after induction by serum deprivation and 1 μM staurosporine. S12 expression significantly increased the rate of apoptosis relative to the control, and S12_{AAA} expression led to a similar result (FIG. 7). Thus, the primary mechanism of S12 acceleration of apoptosis in PC12 cells is not related to the L1145-L1152 segment and not dependent on S12 inhibition of α7nAChR surface expression.

### INCORPORATION BY REFERENCE

Unless stated to the contrary, the entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A recombinant polynucleotide comprising a nucleic acid sequence encoding an engineered SARS-CoV-2 spike protein (S12) or an immunogenic fragment thereof, wherein the engineered spike protein or immunogenic fragment comprises one or more mutations in the S2 segment of the ectodomain.

2. The recombinant polynucleotide of claim 1, wherein the engineered spike protein or immunogenic fragment has a reduced effect on downregulation of surface expression of α7 nicotinic acetylcholine receptor (α7nAChR) in host cells compared to a wild-type SARS-CoV-2 spike protein, or has substantially no effect on α7nAChR surface expression in host cells.

3. The recombinant polynucleotide of claim 1, wherein the engineered spike protein or immunogenic fragment comprises a mutation at one or more positions selected from the group consisting of: L1145, F1148, and L1152, wherein the positions correspond to a wild-type SARS-CoV-2 spike protein or orthologous sites in a variant thereof.

4-6. (canceled)

7. The recombinant polynucleotide of claim 1, wherein the engineered spike protein or immunogenic fragment comprises the amino acid sequence of SEQ ID NO:2 in which at least one amino acid represented by X₁, X₂ and X₃ is mutated relative to the corresponding wild-type amino acid sequence.

8. The recombinant polynucleotide of claim 1, wherein the engineered spike protein or immunogenic fragment comprises an amino acid sequence selected from the group consisting of: SEQ ID NO:3; SEQ ID NO:4; SEQ ID NO:5; SEQ ID NO:6, wherein when X₁ is L, X₂ is not F, and when X₂ is F, X₁ is not L; SEQ ID NO:7, wherein when X₁ is L, X₂ is not L, and when X₂ is L, X₁ is not L; SEQ ID NO:8, wherein when X₁ is F, X₂ is not L, and when X₂ is L, X₁ is not F; SEQ ID NO:9; SEQ ID NO:19, SEQ ID NO:20, SEQ ID NO:21, SEQ ID NO:22, and SEQ ID NO:23.

9-11. (canceled)

12. The recombinant polynucleotide of claim 8 wherein the engineered spike protein or immunogenic fragment comprises the amino acid sequence of SEQ ID NO:9, SEQ ID NO:19, SEQ ID NO:20, SEQ ID NO:21, SEQ ID NO:22, or SEQ ID NO:23.

13-18. (canceled)

19. The recombinant polynucleotide of claim 1, wherein the engineered spike protein or a variant or an immunogenic fragment thereof further comprises proline substitutions at amino acids K986 and V987 relative to the wild-type spike protein or orthologous sites in a variant thereof.

20. The recombinant polynucleotide of claim 1, wherein the nucleic acid sequence comprises a ribonucleic acid (RNA) selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), and any combination(s) thereof.

21. The recombinant polynucleotide of claim 20, wherein the RNA is mRNA.

22. The recombinant polynucleotide of claim 20, wherein the RNA is chemically modified with a pseudouridine.

23. The recombinant polynucleotide of claim 21, wherein the mRNA includes one or more of a stem loop, a chain terminating nucleoside, a polyA sequence, a polyadenylation signal, and/or a 5′ cap structure.

24. The recombinant polynucleotide of claim 1, wherein the engineered spike protein or immunogenic fragment thereof is encoded by a coding sequence, which is codon-optimized and/or the G/C content of which is increased compared to a wild type coding sequence.

25. A polypeptide encoded by the recombinant polynucleotide of claim 1.

26. A cell comprising the recombinant polynucleotide of claim 1.

27. A vaccine comprising:
(a) the recombinant polynucleotide of claim 1; and
(b) a pharmaceutically acceptable carrier.

28-31. (canceled)

32. A pharmaceutical formulation comprising:
(a) the recombinant polynucleotide of claim 1; and
(b) a pharmaceutically acceptable carrier.

33. A method of activating an immune cell against a target antigen, the method comprising exposing the immune cell to the polypeptide of claim 25.

34. A method of treating, alleviating, or managing SARS-CoV-2 infection and/or one or more symptoms thereof in a subject in need thereof, the method comprising administering an effective amount of the vaccine of claim 27.

35-49. (canceled)

50. A recombinant polypeptide comprising:
(a) an amino acid sequence of SEQ ID NO:2 or SEQ ID NO:10; or
(b) an amino acid sequence selected from the group consisting of: SEQ ID NO:3; SEQ ID NO:4; SEQ ID NO:5; SEQ ID NO:6, wherein when X1 is L, X2 is not F, and when X2 is F, X1 is not L; SEQ ID NO:7, wherein when X1 is L, X2 is not L, and when X2 is L, X1 is not L; SEQ ID NO:8, wherein when X1 is F, X2 is not L, and when X2 is L, X1 is not F; SEQ ID NO:9; SEQ ID NO:19; SEQ ID NO:20; SEQ ID NO:21; SEQ ID NO:22; and SEQ ID NO:23.

51-53. (canceled)

54. A method of treating, alleviating, or managing SARS-CoV-2 infection and/or one or more symptoms thereof in a subject in need thereof, the method comprising administering an effective amount of the recombinant polypeptide of claim 50 to the subject.
